(19) United States
(12) Patent Application Publication
(10) Pub. No.: US 2018/0049754 A1
(43) Pub. Date: Feb. 22, 2018

(54) INTERVERTEBRAL PROSTHESIS, APPARATUS FOR IMPLANTING INTERVERTEBRAL PROSTHESES AND SURGICAL METHOD FOR IMPLANTING INTERVERTEBRAL PROSTHESES, PARTICULARLY FOR PERCUTANEOUS MINI-INVASIVE SURGERY PROCEDURES

(71) Applicant: REDEMED S.R.L., Lucca (LU) (IT)

(72) Inventors: Luigi TARRICONE, Surbo (IT); Luca ANTONELLI, Lecce (IT)

(73) Assignee: REDEMED S.R.L., Lucca (LU) (IT)

(21) Appl. No.: 15/557,913

(22) PCT Filed: Mar. 11, 2016
(86) PCT No.: PCT/EP2016/055346

§ 371 Date: Sep. 13, 2017

Related U.S. Application Data
(63) Continuation-in-part of application No. 14/657,803, filed on Mar. 13, 2015.

(51) Int. Cl. A61B 17/16; A61F 2/44; A61F 2/46

(52) U.S. Cl. CPC A61B 17/1671; A61B 17/1659; A61F 2/442; A61F 2/4465; A61F 2/4611; A61F 2002/4629; A61F 2002/4475; A61F 2002/4627; A61F 2002/4635; A61F 2002/4661; A61F 2/4657

(57) ABSTRACT

An intervertebral prosthesis is provided that has a substantially disc-like geometry with a thickness equal to the intervertebral distance to be restored. In a lateral elevation view, in the intervertebral prosthesis there is a through hole adapted to allow its sliding along a guiding wire inserted previously in the patient through a percutaneous mini-invasive anterolateral access. The apparatus used to perform the implantation consists of an operating table, on which the patient is rested, preferably in a prone position, provided with a radiological device adapted to take snapshots of radiographs from various angles to monitor the surgical procedures inside the patient, as well as an articulated arm that supports a guiding element adapted to allow correct driving of the wire-guided surgical instruments required for the surgical procedure.

The present invention relates to an intervertebral prosthesis, particularly for percutaneous mini-invasive surgery procedures, and an apparatus and surgical method for implanting said intervertebral prosthesis.

Numerous disorders affecting the spinal column and in particular affecting the intervertebral discs are currently known in medicine.

Some of these disorders produce a degeneration of the fibrous capsule of the intervertebral disc, which reduces its elasticity and becomes damaged, allowing part of the nucleus pulposus to escape.

This phenomenon is commonly known by the term “hernia”.

There are also other disorders, of a progressive type, which entail the thinning, over time, of the intervertebral disc due to the loss of its ability to retain water inside it. This thinning often also produces the thinning of the facet capsule, with consequent pain.

In some cases, this thinning can read to reduction of the medullary canal, with a severe risk of chronic claudication.

In many cases, progressive deterioration of the intervertebral disc requires the implantation of an intervertebral prosthesis, which substantially replaces said disc.

Among the various known types of intervertebral prosthesis, one which has the main function of facilitating the fusion of two adjacent vertebrae is currently available.

This type of prosthesis comprises generally one or two bodies made of osteoconductive material which are arranged, by means of a surgical procedure, in mutually opposite positions between the two adjacent vertebrae.

The materials used to provide the two bodies facilitate the growth and adhesion of bone tissue so as to cause the fusion of the two vertebrae, which can no longer move with respect to the other.

This type of intervertebral prosthesis of a known kind, which is generally applied at the level of the lumbar vertebrae, which are the ones subjected to the greatest load caused by body weight, is not free from drawbacks, which include the fact that generally it requires an extremely invasive surgical procedure for implantation.

The surgeon in fact proceeds by preparing the intervertebral disc to accommodate the intervertebral prosthesis and inserts the prosthesis itself by creating initially a percutaneous anterolateral access of such size as to be able to accommodate a cylindrical retractor instrument, which once inserted in the patient through said percutaneous anterolateral access allows to have a maneuvering channel that has a width comprised generally between six and nine centimeters, is delimited laterally by the retractor instrument and is fully free from the organic tissues that are present between the access created and the intervertebral disc to be operated.

In this manner, the surgeon can operate on the disc by working visually and by inserting the various surgical instruments, as well as the prosthesis itself, through the maneuvering channel that has been created.

In greater detail, the method described above provides for entry with a first small cylindrical instrument, then with cannulas that have the function of expanding the first access, and finally with a retractor, which also expands further the first access.

It should be stressed that this surgical procedure, in addition to being inherently laborious and time-consuming, can lead to severe consequences for the patient, since although the procedure is monitored at the neurological level by a device that detects the presence of a nervous structure proximate to the surgical instrument, it does not allow to detect and therefore monitor the compression of tissues and muscles (against the transverse apophyses) caused by the divarication or expansion of the retractor instrument, which very often leads to the stretching and/or compression of the femoral plexus throughout the duration of the procedure.

This can lead sometimes to temporary dysesthesia of the associated femoral nerve and/or to temporary paresthesias, to temporary motor deficits of the quadriceps, to temporary weakness in hip flexion, and in some cases to actual permanent damage of said plexus, all the consequences cited above being permanent.

Indeed, in recent times, manufacturers of intervertebral prostheses recommend closing the retractor every 10 minutes, waiting just as long to then resume the procedure.

Another drawback of the background art, discussed in some studies, resides in that in a lateral position at the L4-L5 and L3-L4 level the veins and aortas move closer to the space affected by the procedure.

A further drawback of the background art resides in that it requires the removal also of a portion of anulus that is as wide as the implant that will be positioned in addition to the internal parts of the disc (nucleus pulposus), which leads to an incorrect placement of the implant in said disc.

A further drawback of the background art resides in the onset, a short time later, of inguinal hernias caused by access (tissue stress).

A further drawback of the background art resides in that if it is necessary to remove the implant due to infections, incorrect placement or size, et cetera, surgery to remove and/or replace the implant is highly invasive and complex.

A further drawback of the background art resides in that if it is necessary to provide posterior stabilization (70% of cases), with the background art first of all lateral access is provided in order to position the implant and then the patient is turned over to perform the arthrodesis procedure via a posterior pathway; this entails removing all the surgical sheets from the patient, repositioning him on the operating table, placing again the new sheets with the posterior access and continuing the procedure. If efficient operating room (O.R.) staff is available, this procedure requires 25 minutes, extending all operating times (anesthesia, etc.).

The aim of the present invention is to provide an intervertebral prosthesis that is adapted to create a bone bridge between two adjacent vertebrae to be fused, such that it can be implanted in full safety with a percutaneous and mini-invasive procedure, so as to overcome the limitations and drawbacks of the background art.

Within the scope of this aim, an object of the present invention is to provide a surgical method and to provide an apparatus that allow the implantation of said intervertebral prosthesis in a manner that is simple, fast, effective and most of all reliable.

This aim, as well as these and other objects that will become better apparent hereinafter, are achieved by an intervertebral prosthesis, particularly for percutaneous mini-invasive surgery procedures, comprising an element that has a substantially disc-like shape and is adapted to be inserted between two adjacent vertebrae of a patient in which said intervertebral prosthesis is to be implanted as a replacement of the intervertebral disc comprised between them, so as to support entirely the vertebral endplates over the largest possible surface and at the same time impart an anteroposterior angle aimed at maintaining physiological lordosis and sagittal balance in order to form a bone bridge between said adjacent vertebrae, characterized in that said element has a through hole that passes through said element from side to side and is adapted to accommodate slidingly a guiding wire, inserted beforehand in said patient along a direction that is perpendicular to the sagittal plane of said patient through a percutaneous anterolateral access, for the wire-guided insertion of said intervertebral prosthesis, said through hole being extended along a radial direction with respect to the geometry of said element so that it is oriented, once implanted, along a direction that is substantially perpendicular to the craniocaudal axis of said patient and to said sagittal plane.

Furthermore, this aim, as well as these and other objects that will become better apparent hereinafter, are achieved by an apparatus for implanting intervertebral prostheses, particularly for percutaneous mini-invasive surgery procedures, comprising:
an operating table on which the patient to be operated is rested,
surgical instruments required for the surgical procedure,
a radiological device adapted to take snapshots of radiographs in order to determine the exact position of the intervertebral disc on which to operate and the operating trajectory for guiding said surgical instruments by checking for the presence of intestinal loops or loops of the peritoneum along said operating trajectory so as to avoid the tearing or passing through thereof,

characterized in that it comprises at least one guiding wire that can be inserted in said patient through a percutaneous anterolateral access along a direction that is substantially perpendicular to the craniocaudal axis and to the sagittal plane of said patient, said surgical instruments being slidingly associable with said guiding wire to perform the surgical procedure in a wire-guided manner.

Furthermore, this aim, as well as these and other objects that will become better apparent hereinafter, are achieved by a surgical method for implanting intervertebral prostheses, particularly for percutaneous mini-invasive surgery procedures, comprising:
placing a patient to be operated on an operating table,
taking a first radiograph by means of a radiological device in order to establish the exact position of the intervertebral disc to be operated,
opening a percutaneous anterolateral access by means of a scalpel,
inserting in said patient, through said percutaneous anterolateral access, a series of surgical instruments adapted to prepare said intervertebral disc to accommodate an intervertebral prosthesis,
inserting said intervertebral prosthesis in said patient through said percutaneous anterolateral access,

characterized in that said insertion steps are performed with the aid of a guiding wire, inserted previously in said patient through said percutaneous anterolateral access along a direction that is substantially perpendicular to the craniocaudal axis and to the sagittal plane of said patient, said surgical instruments and said intervertebral prosthesis being associable slidingly with said guiding wire to perform the surgical procedure in a wire-guided manner.

Further characteristics and advantages of the invention will become better apparent from the description of two preferred but not exclusive embodiments of an intervertebral prosthesis, of an apparatus for implanting intervertebral prostheses and of a surgical method for implanting intervertebral prostheses, particularly for percutaneous mini-invasive surgery procedures, according to the invention, illustrated by way of nonlimiting example in the accompanying drawings, wherein:

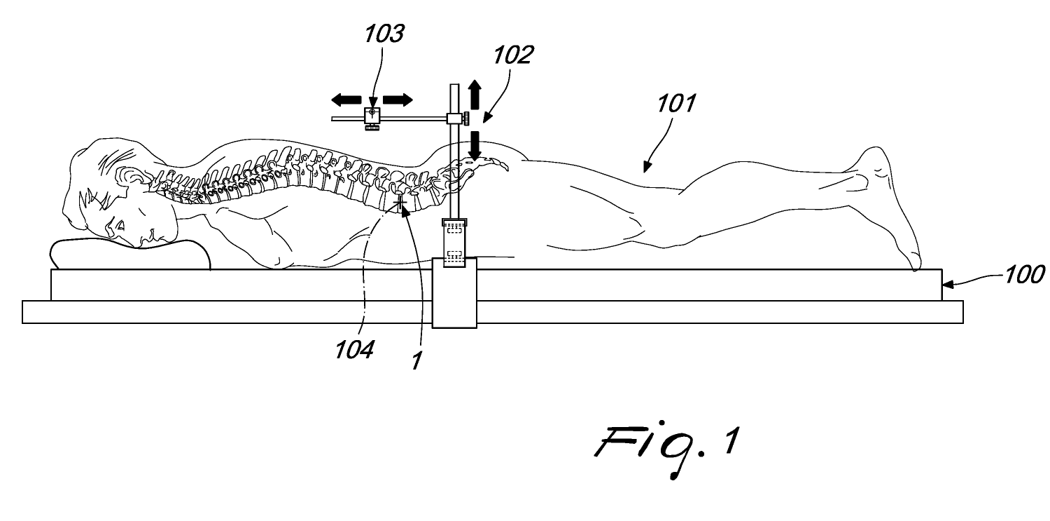

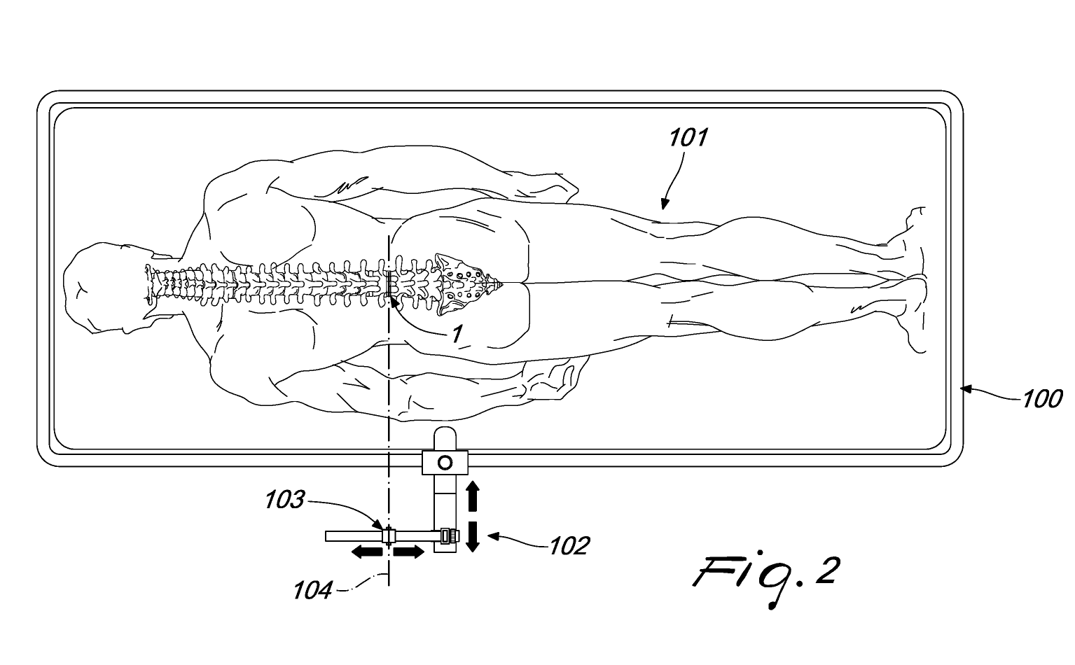

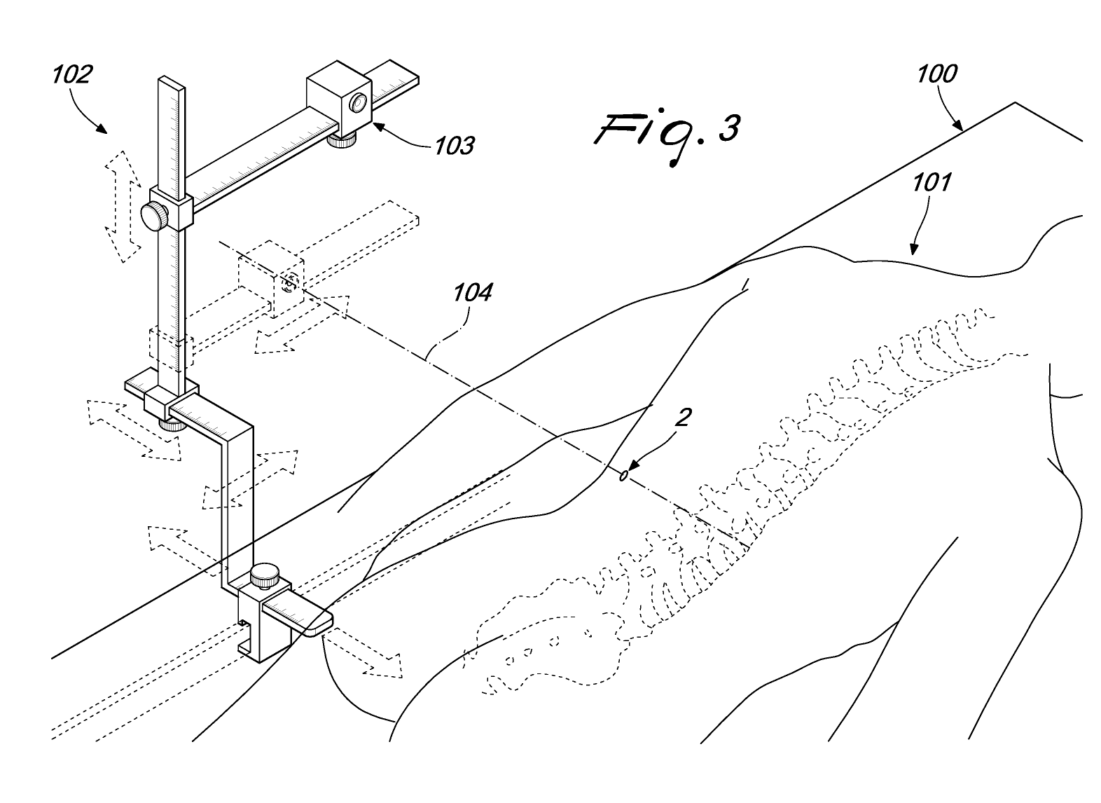

FIGS. 1 to 3 are three views, respectively a lateral elevation view, a top plan view and a perspective view, of a schematic representation of the articulated arm that supports the guiding element, according to the present invention, during its positioning with respect to a patient lying prone on an operating table;

FIG. 4 is a lateral elevation view of the articulated arm, shown in the preceding figures, after positioning has been performed with respect to a patient lying prone on the operating table;

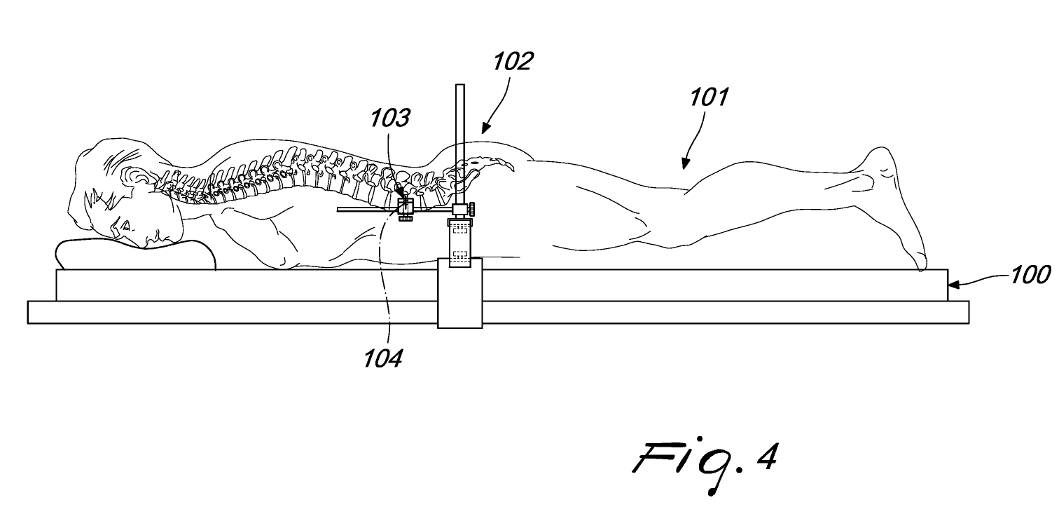

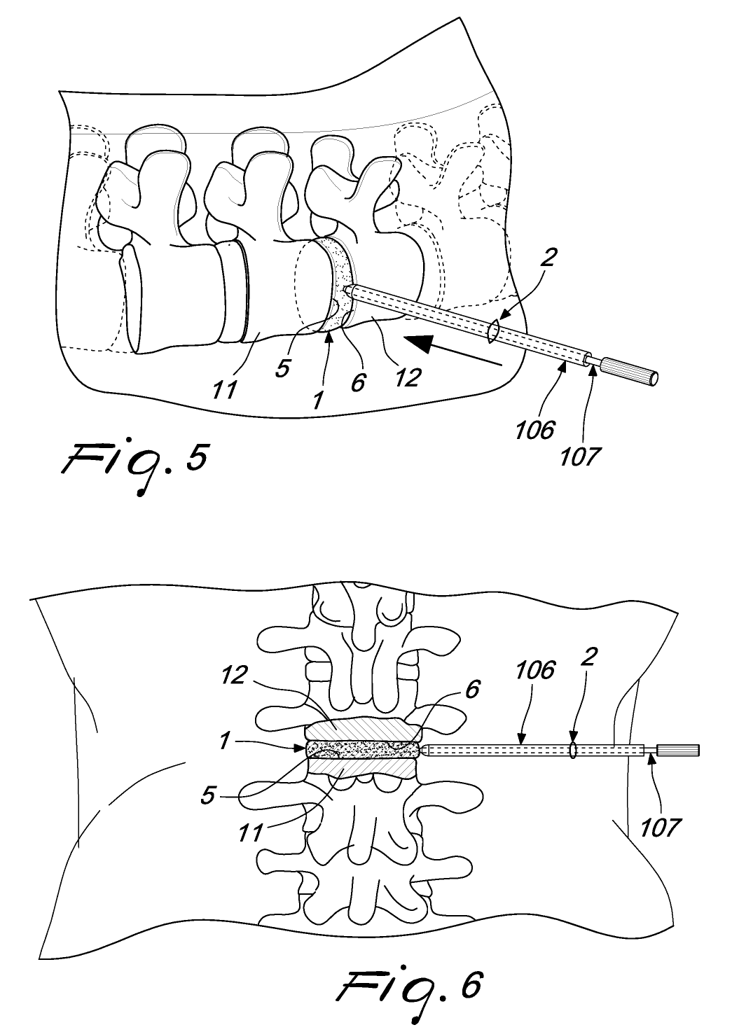

FIGS. 5 and 6 are two views, respectively a perspective view and a top plan view, of a representation of the step of insertion of a cannulated instrument provided with a plug element, according to the present invention;

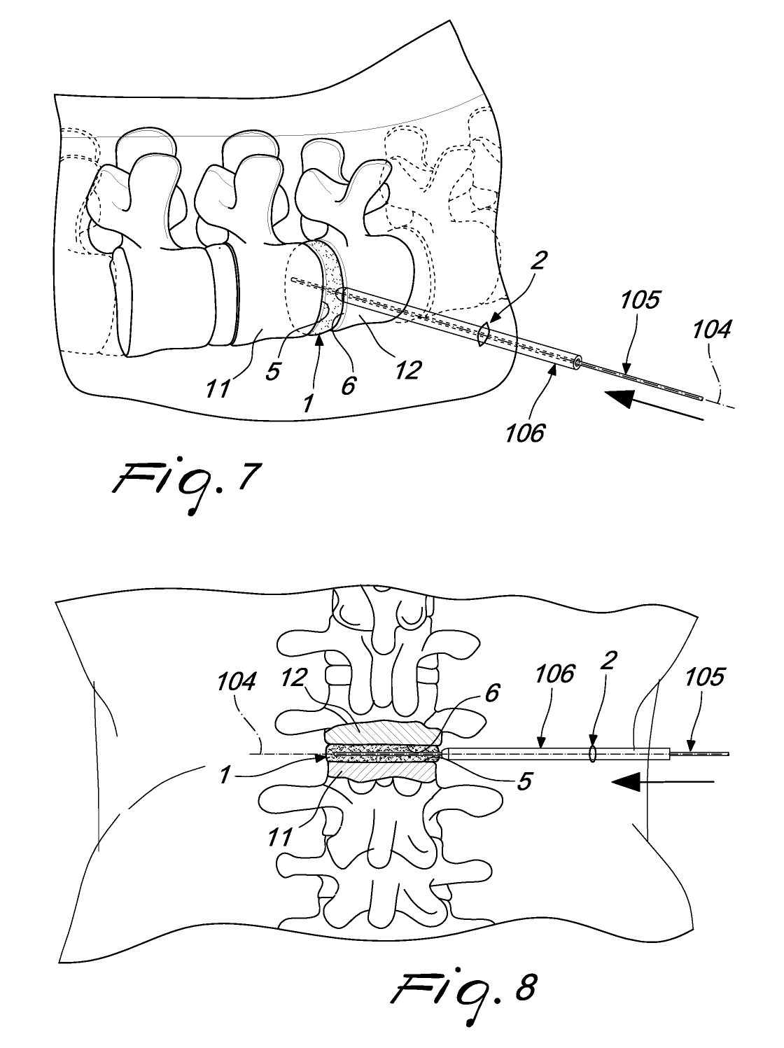

FIGS. 7 and 8 are two views, respectively a perspective view and a top plan view, of a representation of the step of insertion of a guiding wire within the cannulated instrument, according to the present invention, until the intervertebral disc to be operated is penetrated;

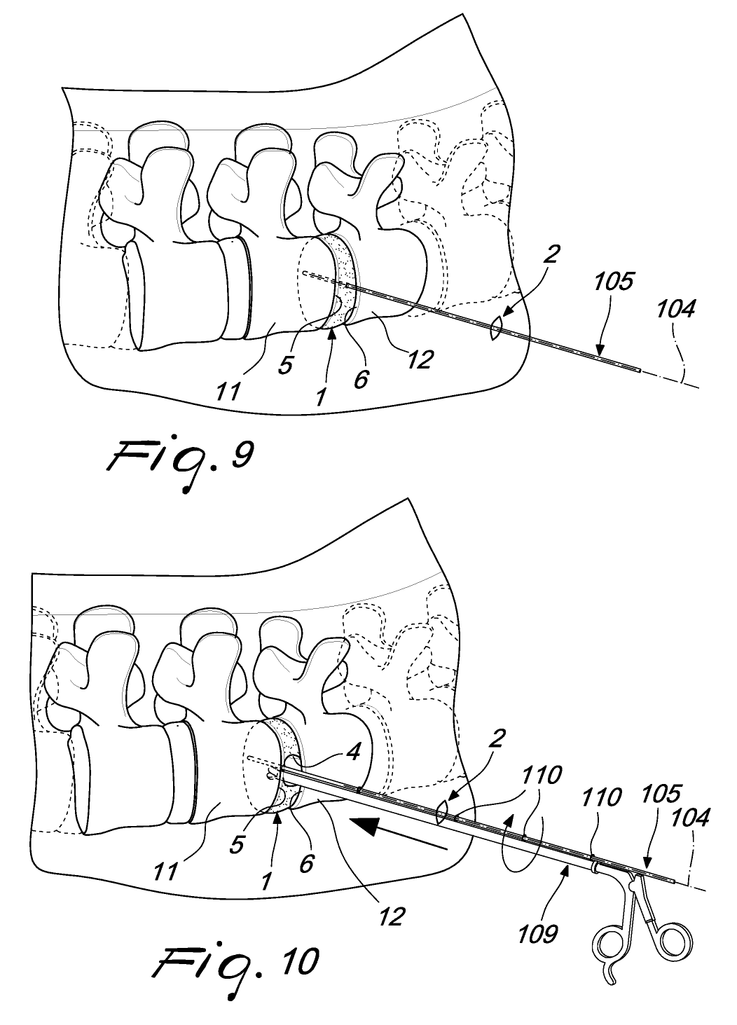

FIG. 9 is a perspective view of the guiding wire, shown in the preceding figures, positioned inside the patient, according to the present invention, once the cannulated instrument has been removed;

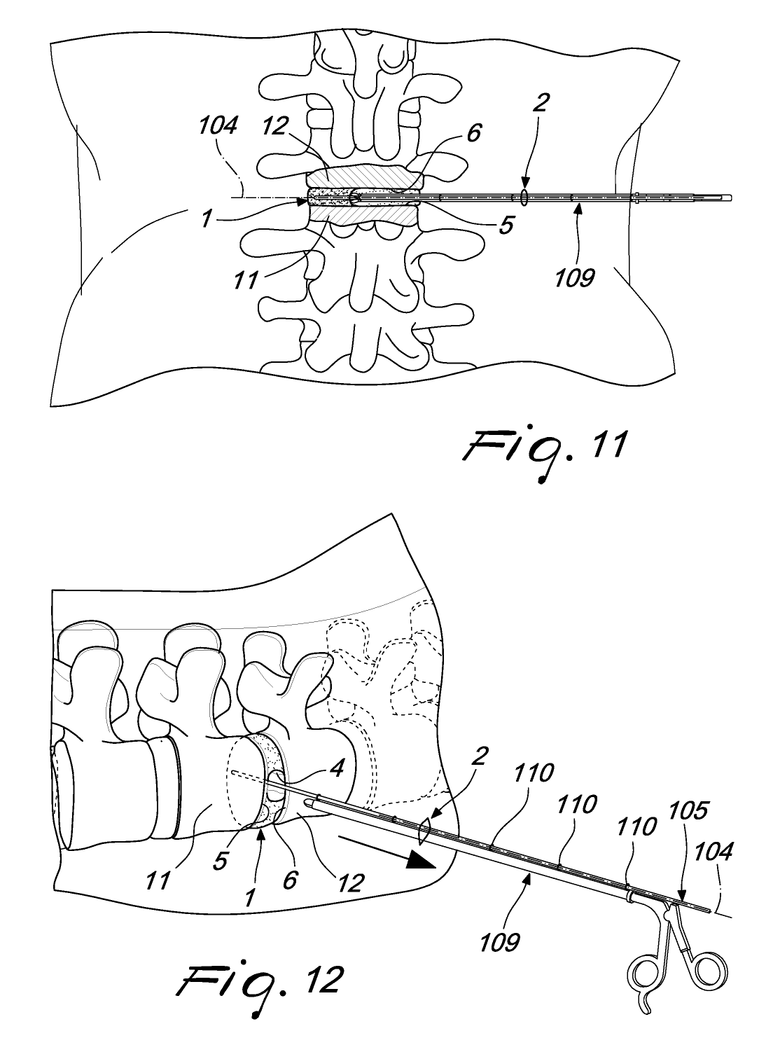

FIGS. 10 to 12 are three views, respectively a perspective view, a top plan view and again a perspective view, of a representation of the step of wire-guided insertion of a hernia clamp and of the step of removal of part of the intervertebral disc, according to the present invention;

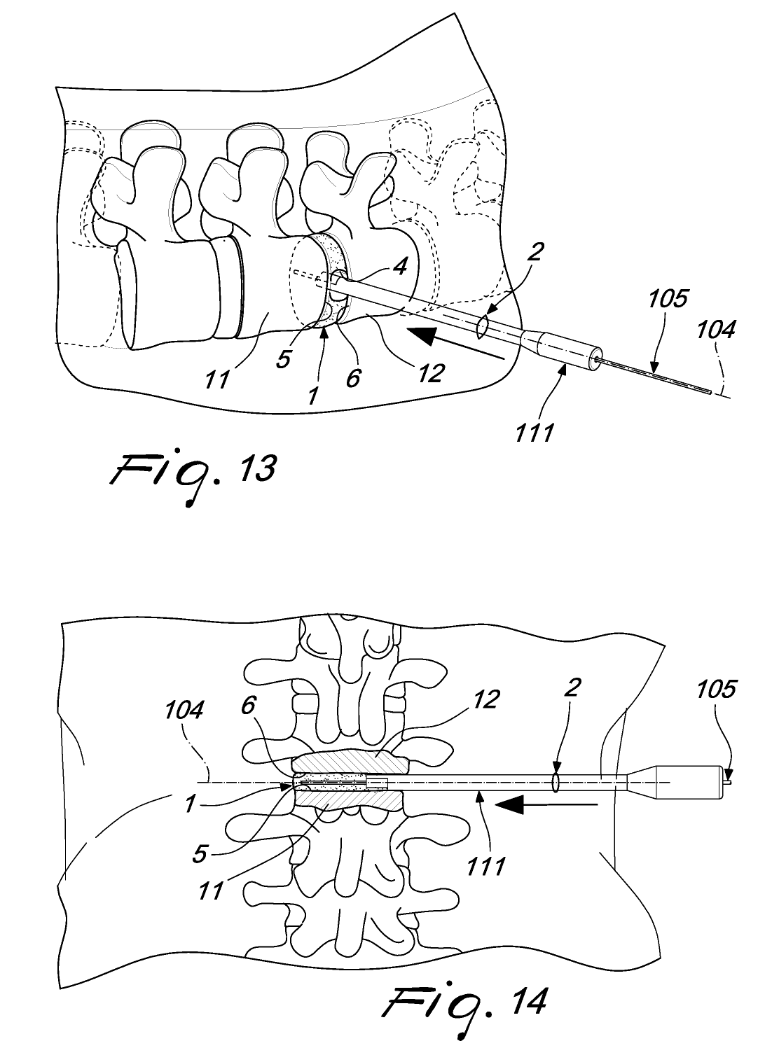

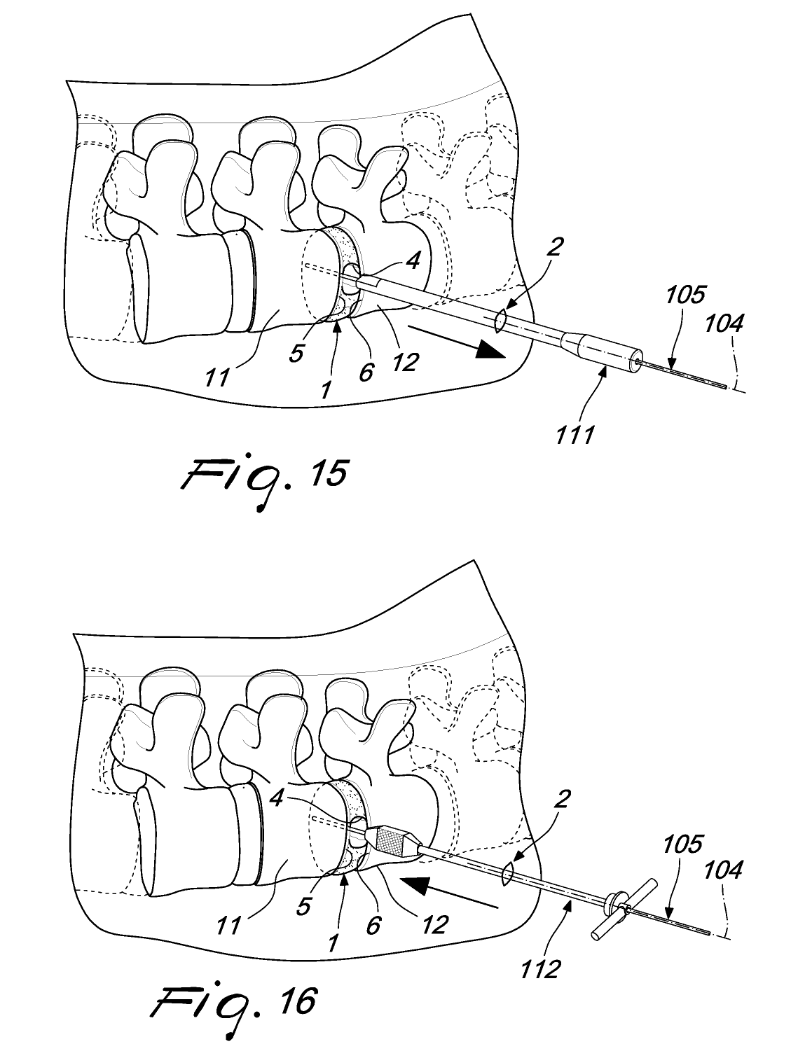

FIGS. 13 to 15 are three views, respectively a perspective view, a top plan view and again a perspective view, of a representation of the step of wire-guided insertion of a cannulated rasp and of the step of removal of the cartilage of the vertebral endplates with bleeding thereof, according to the present invention;

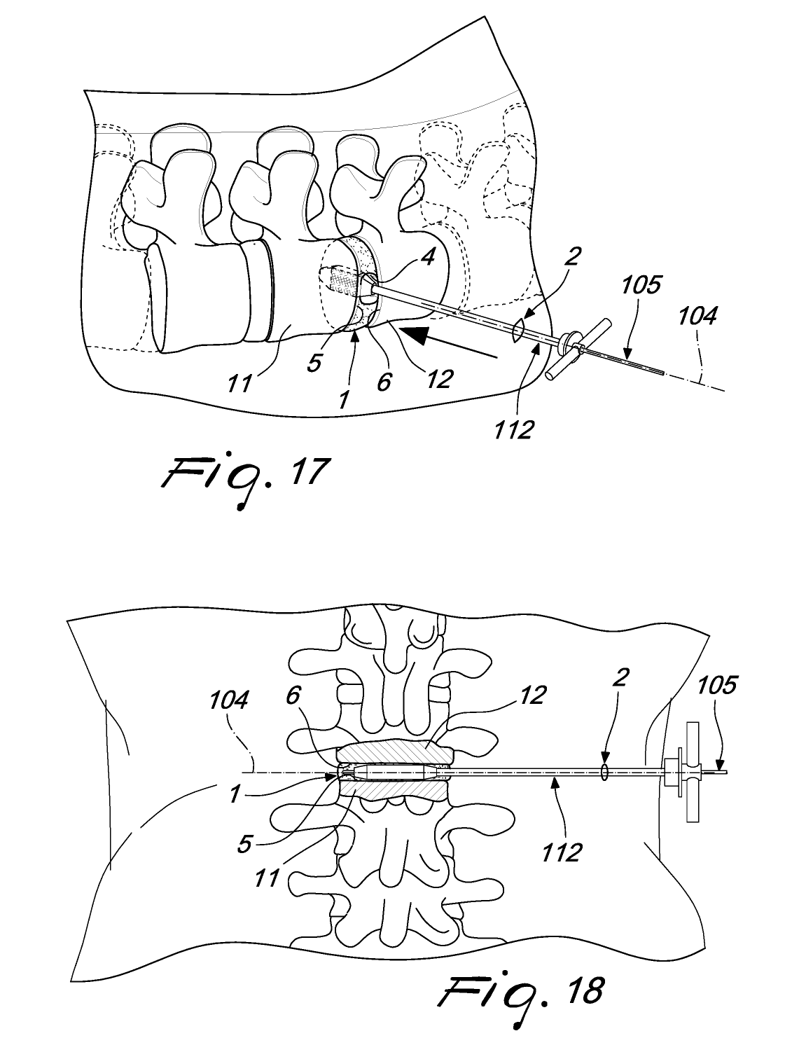

FIGS. 16 to 18 are three views, respectively a perspective view, a top plan view and again a perspective view, of a representation of the step of wire-guided insertion of a cannulated measurer within the intervertebral disc in order to determine the height of the intervertebral prosthesis to be implanted;

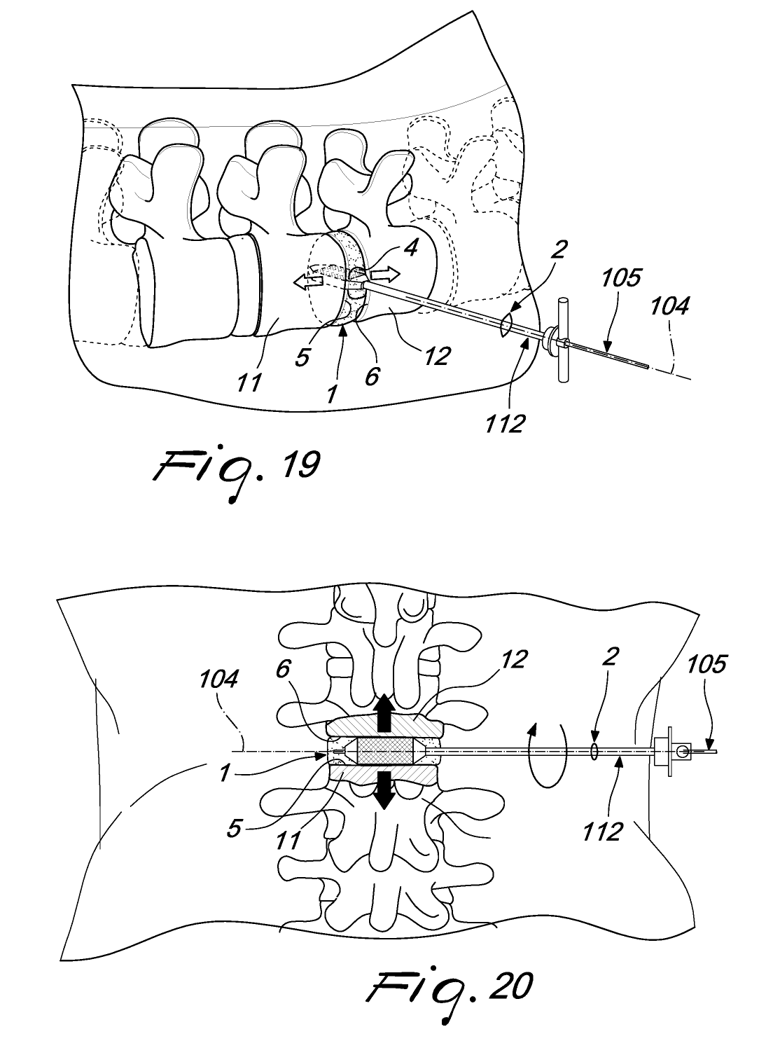

FIGS. 19 and 20 are two views, respectively a perspective view and a top plan view, of a representation of the step of restoring the intervertebral space between the intervertebral endplates, according to the present invention, by means of the cannulated measurer shown in the preceding figures;

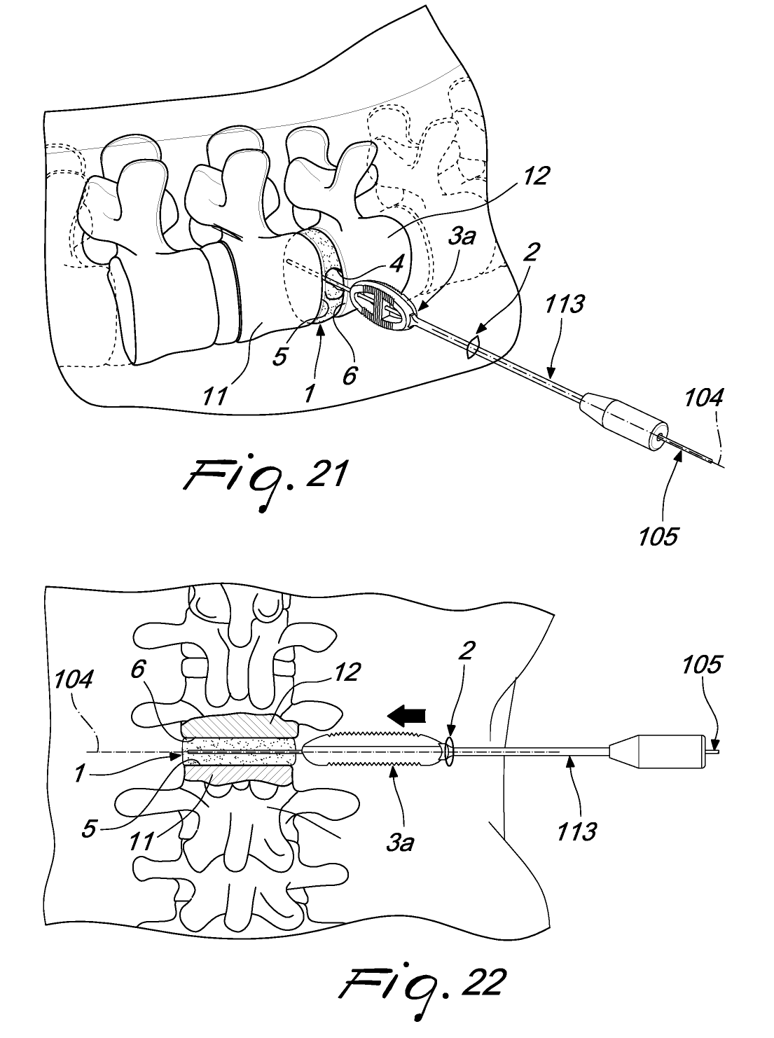

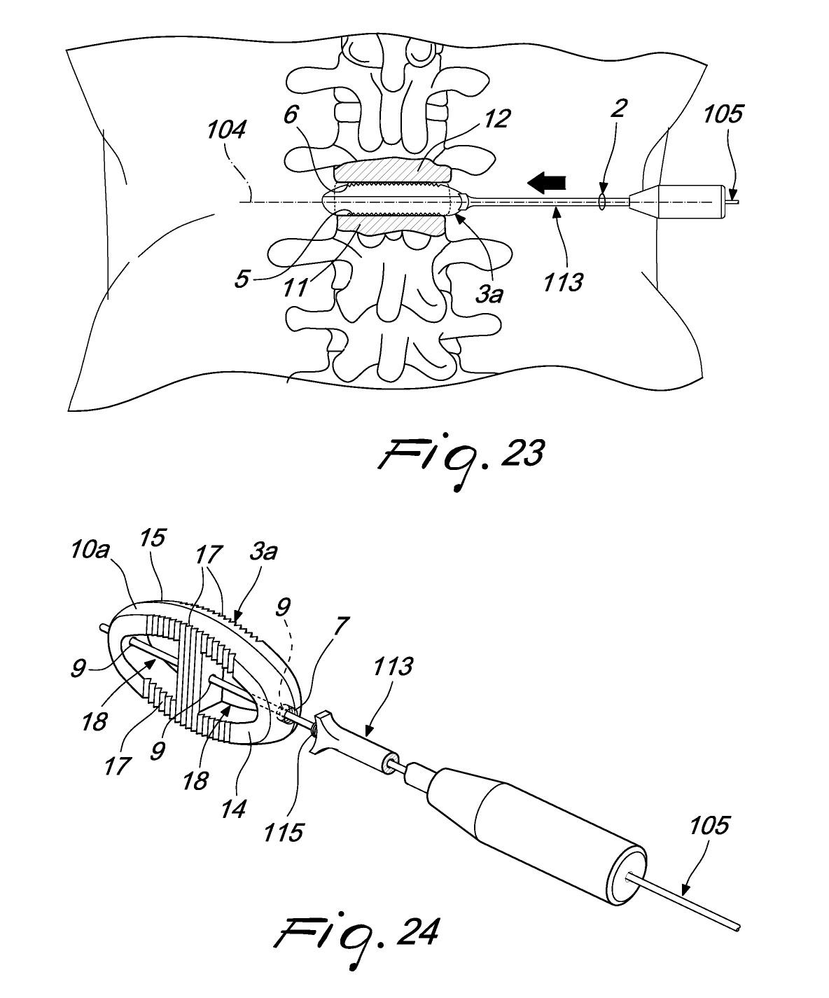

FIGS. 21 to 23 are three views, of which the first one is a perspective view and the subsequent ones are plan views, of a representation of the step of wire-guided insertion of a first embodiment of an intervertebral prosthesis, according to the present invention, by means of an insertion instrument;

FIG. 24 is an exploded perspective view of the intervertebral prosthesis shown in the preceding FIGS. 21 to 23 and of part of the inserted instrument, according to the present invention;

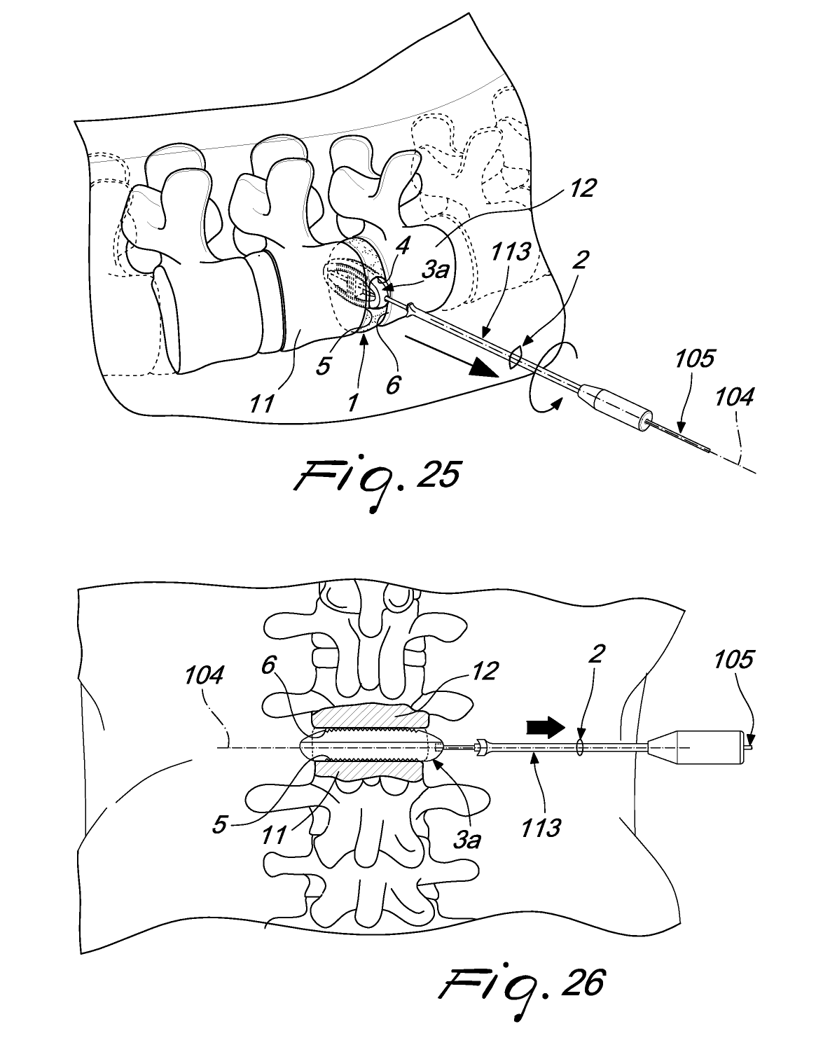

FIGS. 25 and 26 are two views, respectively a perspective view and a top plan view, of a representation of the step of extraction of the cannulated instrument once the intervertebral prosthesis has been positioned, according to the present invention;

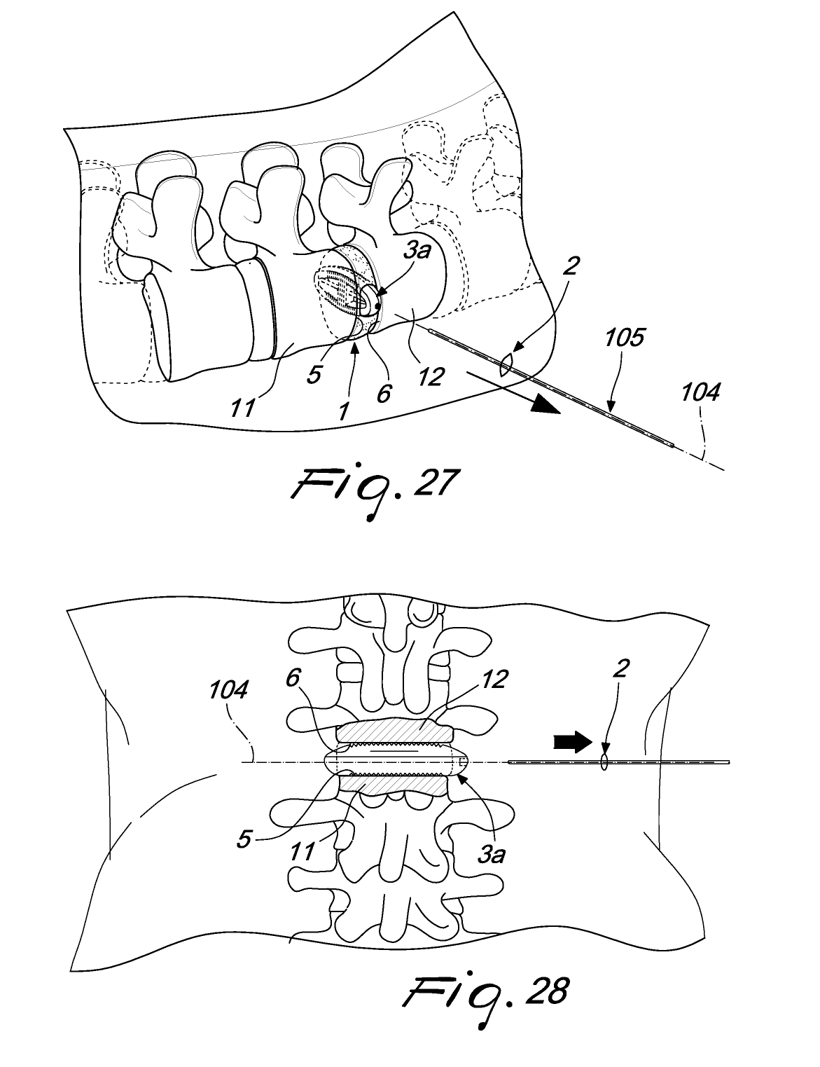

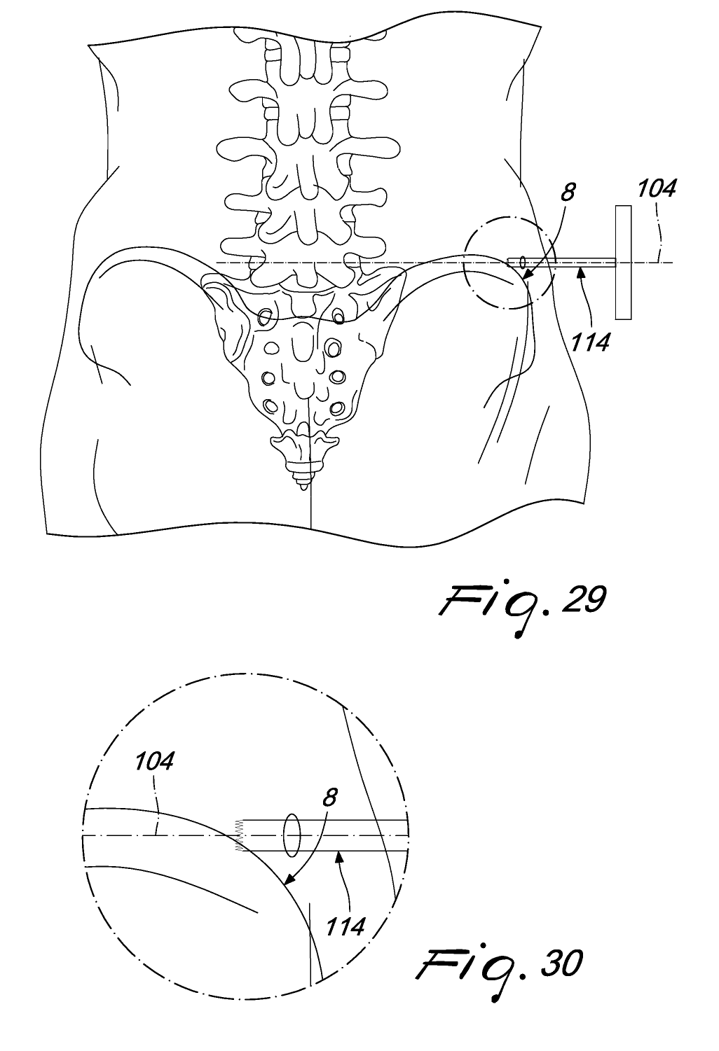

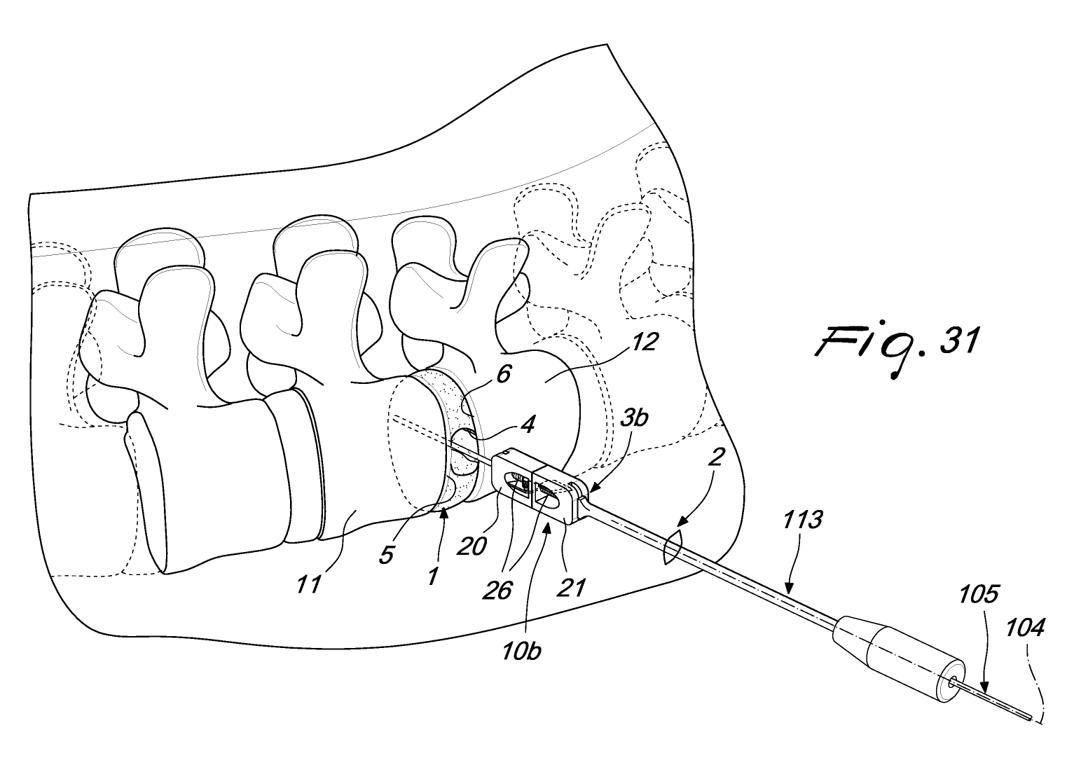

FIGS. 27 and 28 are two views, respectively a perspective view and a top plan view, of a representation of the step of extraction of the guiding wire once the intervertebral prosthesis has been positioned, according to the present invention;

FIG. 29 is a top plan view of a representation of the milling step of a milling tool, in a manner that is guided by means of the guiding element shown in the preceding figures, to create a passage through which said cannulated instrument is to be inserted;

FIG. 30 is an enlarged-scale view of a detail of the milling tool shown in FIG. 29;

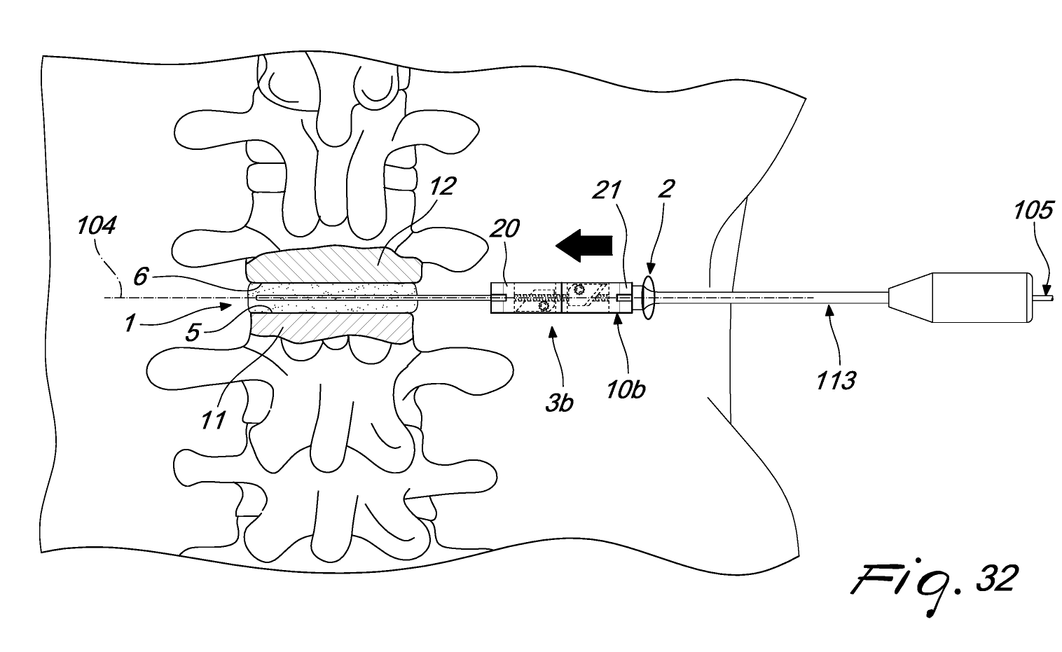

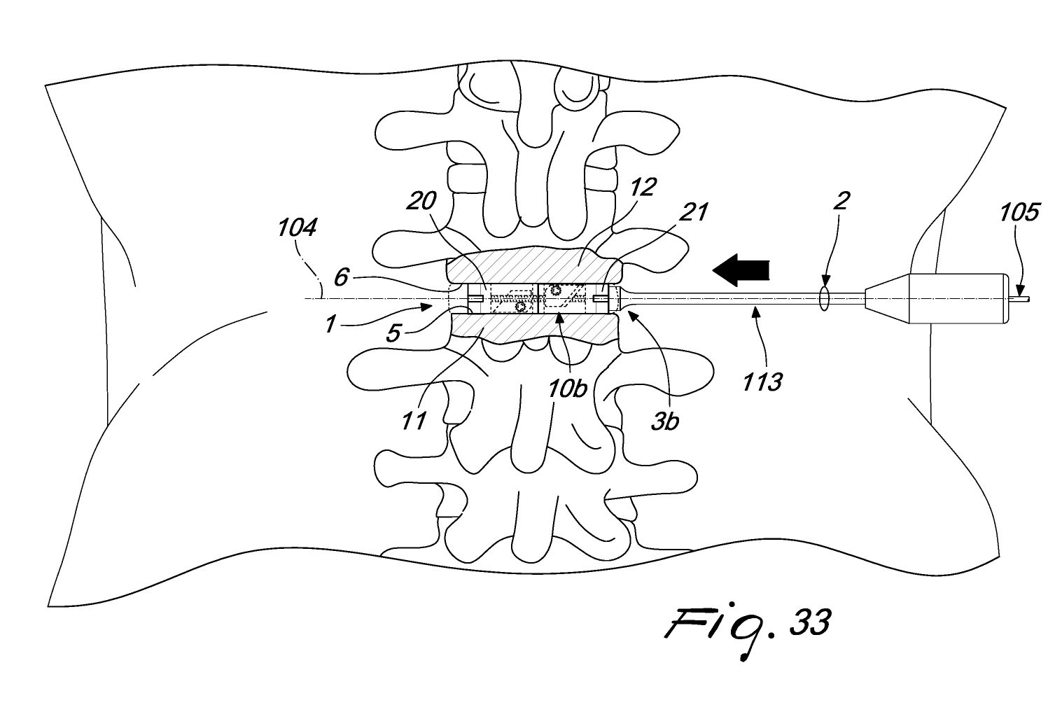

FIGS. 31 to 33 are three views, of which the first one is a perspective view and the subsequent ones are plan views, of a representation of the step of wire-guided insertion of a second embodiment of an intervertebral prosthesis, according to the present invention, by means of said insertion instrument;

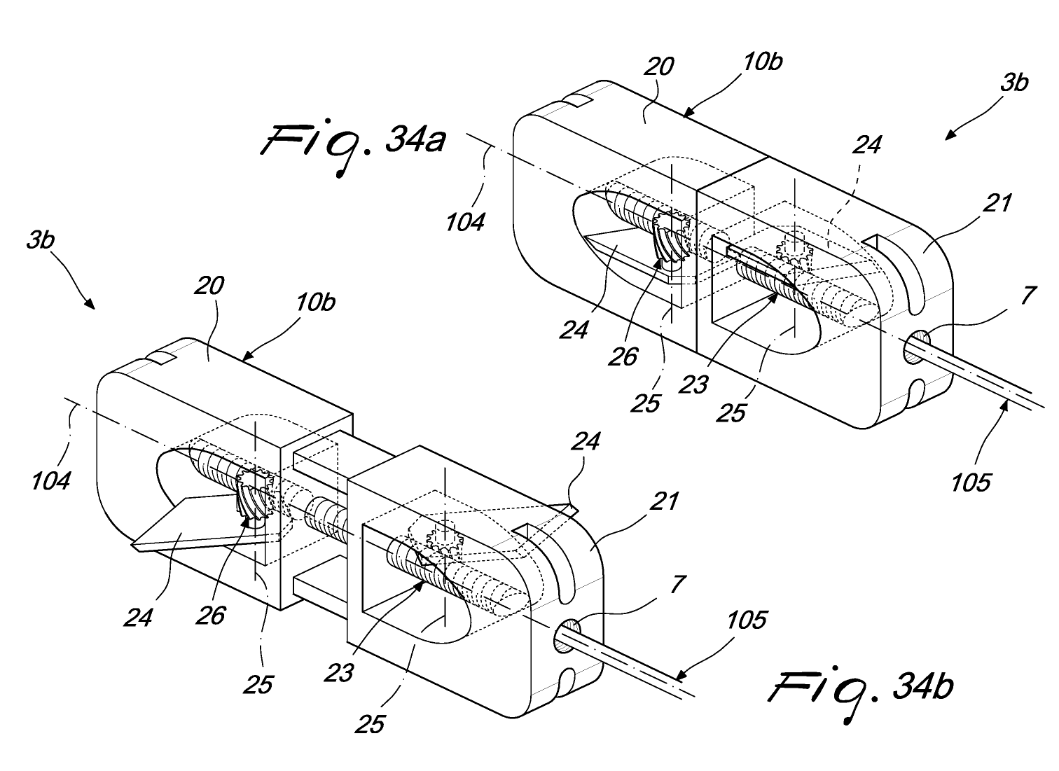

FIGS. 34a and 34b are two perspective views of the intervertebral prosthesis shown in the previous FIGS. 31 to 33, respectively, in two different configurations of operation thereof;

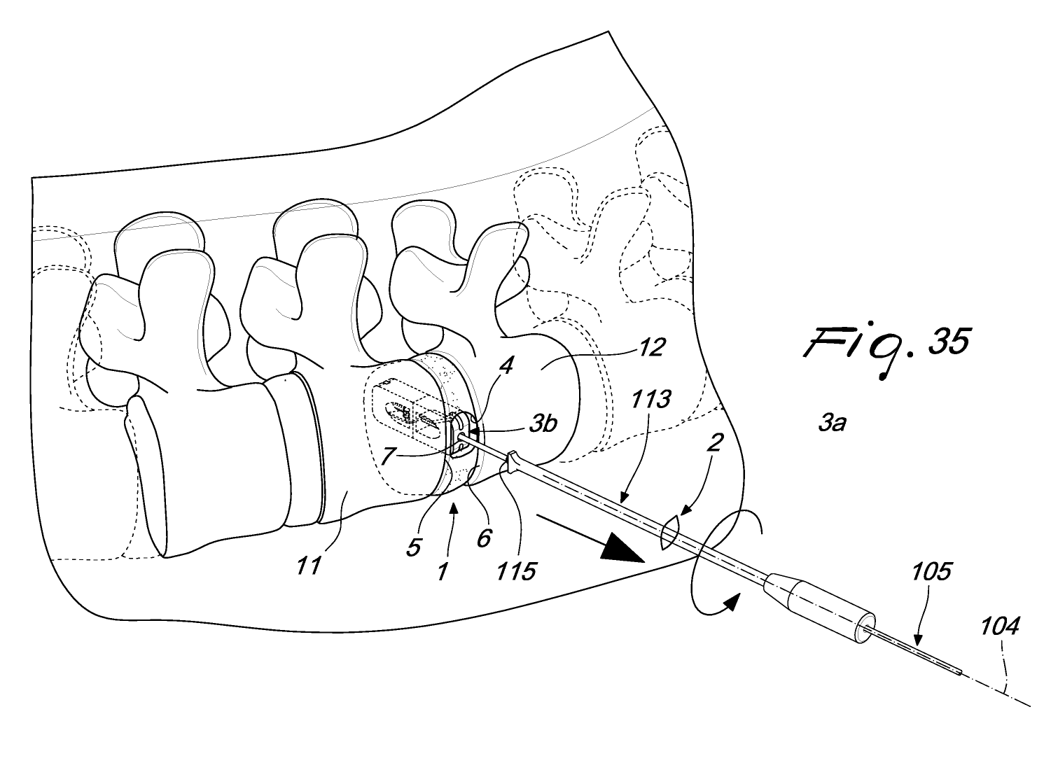

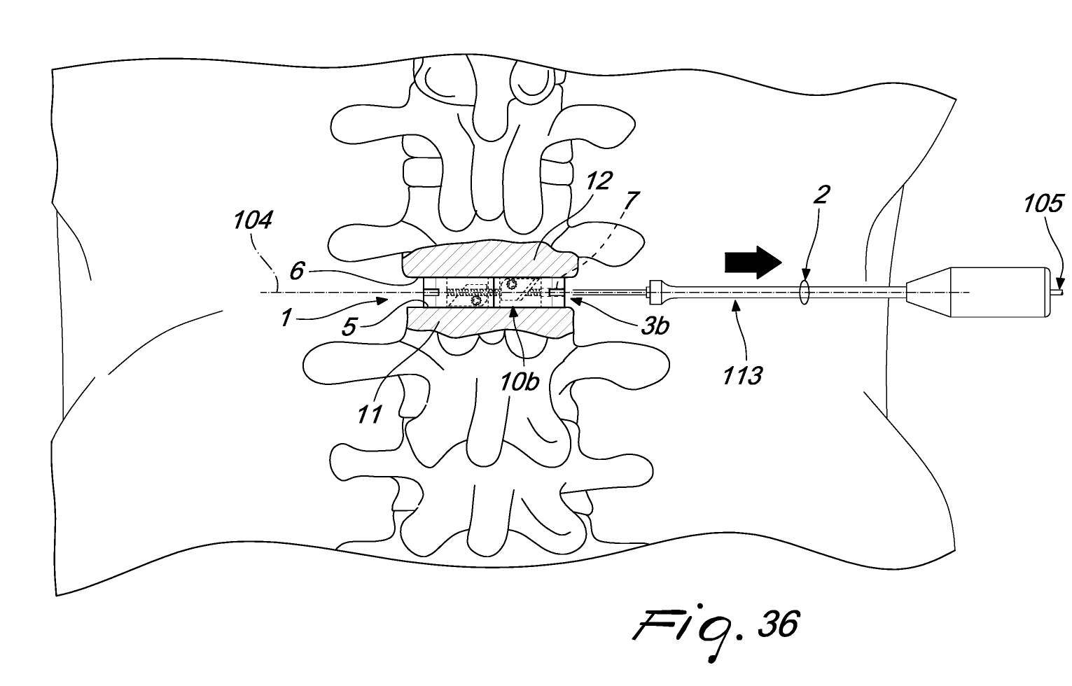

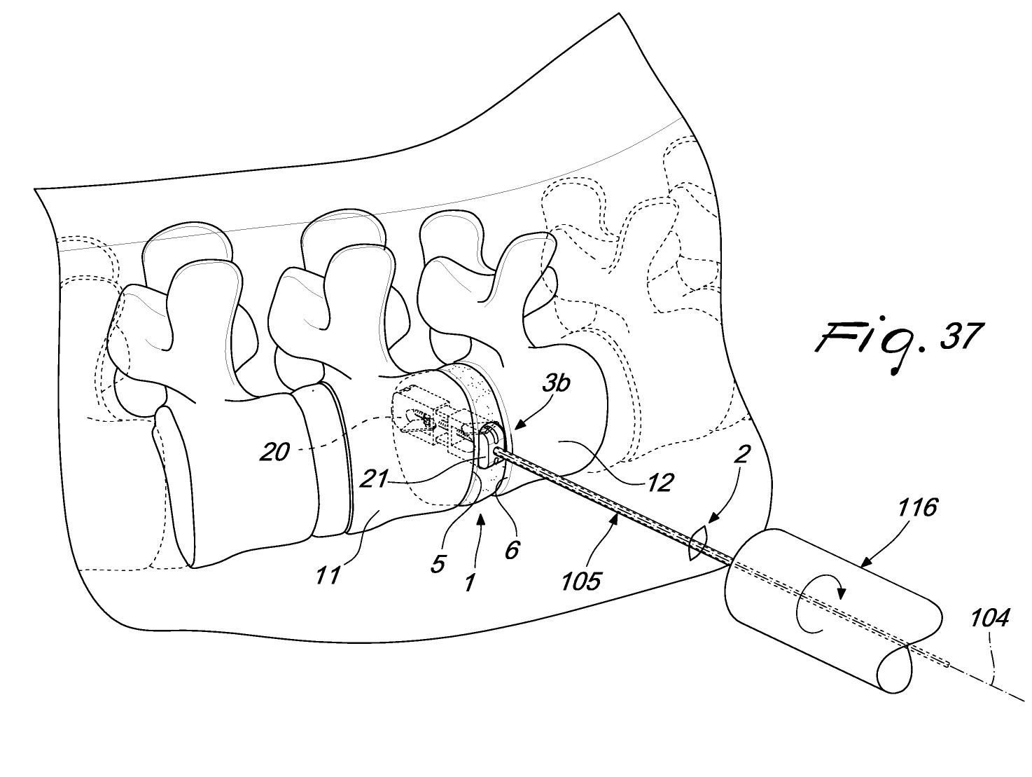

FIGS. 35 and 36 are two views, respectively a perspective view and a top plan view, of a representation of the step of extraction of the cannulated instrument once the intervertebral prosthesis has been positioned, according to the present invention;

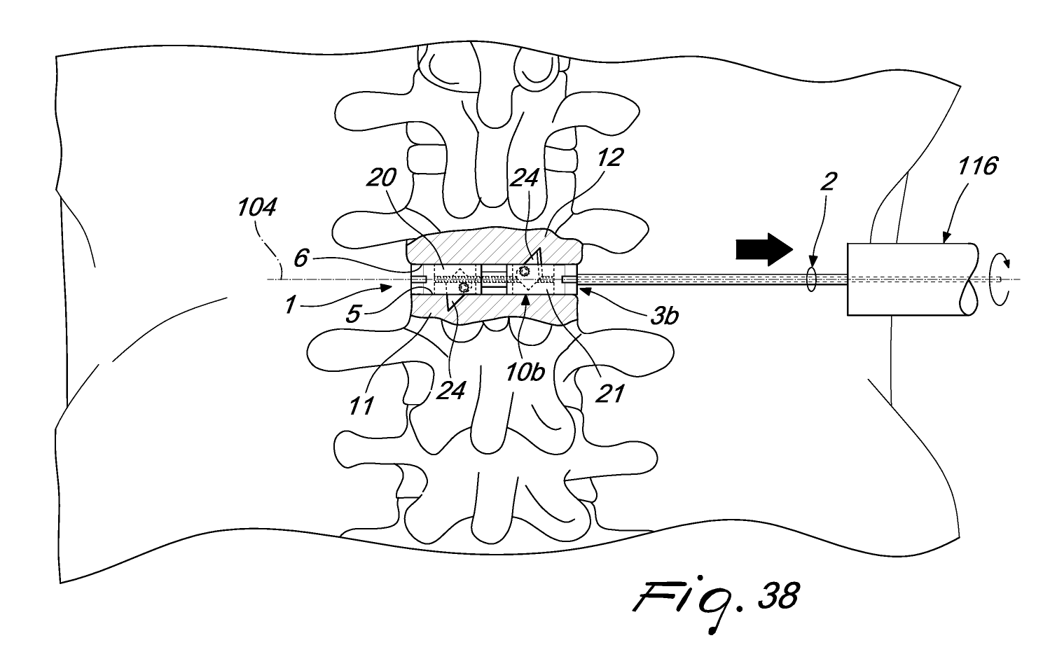

FIGS. 37 and 38 are two views, respectively, a perspective view and a plan view from above, of a representation of the step of expansion and of stabilization of the intervertebral prosthesis, according to the present invention;

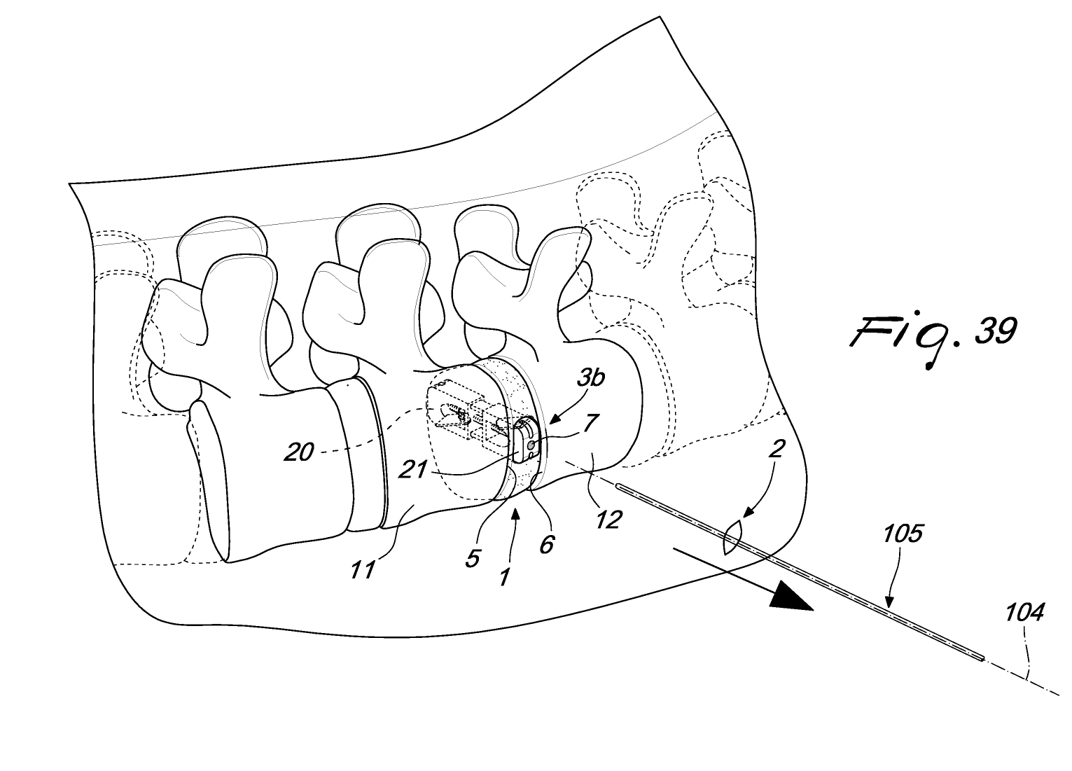

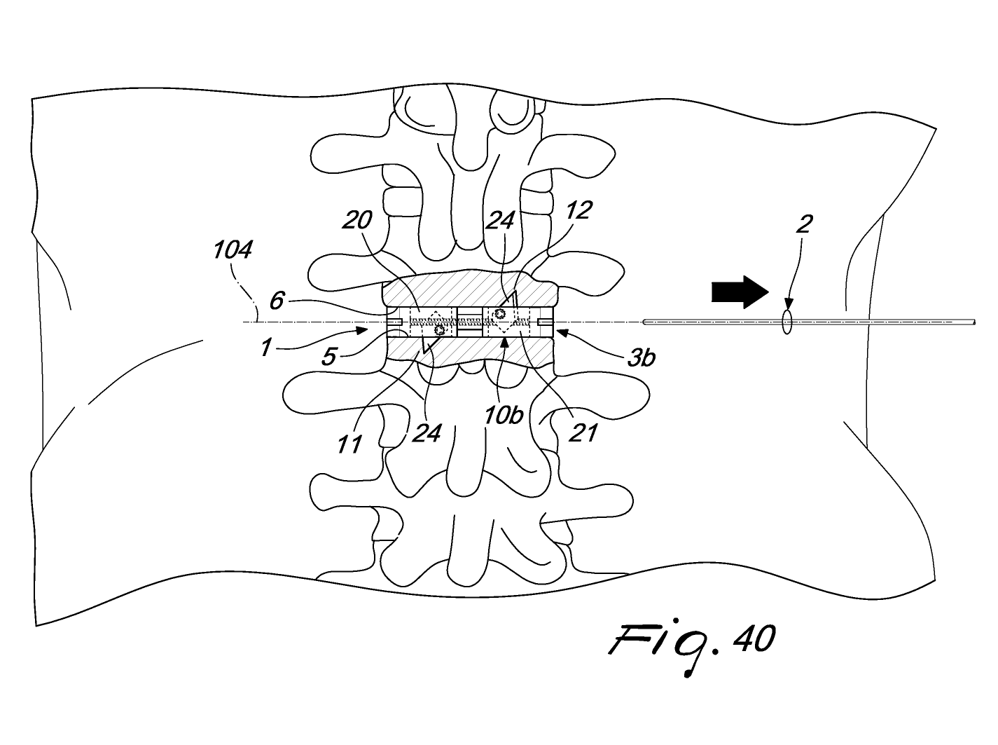

FIGS. 39 and 40 are two views, respectively a perspective view and a top plan view, of a representation of the step of extraction of the guiding wire once the intervertebral prosthesis has been positioned, according to the present invention.

With reference to the cited figures, the surgical method for implanting intervertebral prostheses, particularly for percutaneous mini-invasive surgery procedures, comprises first of all the placement on an operating table 100 of a patient 101 to be operated, preferably in prone position.

Then a first radiograph is performed by means of a radiological device, not shown for the sake of graphical simplicity, in order to establish the exact position of the intervertebral disc 1 to be operated.

This radiological device, which is per se known and therefore is not described in detail, can comprise for example an image intensifier with a video post or a C-shaped arch with which multiple radiological snapshots from multiple angles are taken.

In order to check for the presence of intestinal loops or of loops of the peritoneum along the optimal direction in which the implant will be carried out, there can be a further step of pre-operative radiology, carried out for example by means of a CAT (acronym of Computerized Axial Tomography) scan, to be performed before surgery thus defining the operating trajectory 104 along which to guide the surgical instruments for the implant thus preventing the tearing or passing through of such soft tissues and, as a consequence, thus avoiding the extremely serious complications that damage of this type can cause to the patient 101.

Once the point to be operated has been identified, as shown in FIGS. 1 to 4, an articulated arm 102, for example of the motorized type or the manual type with sliders, is positioned with respect to the operating table 100 so as to arrange a guiding element 103, with which the articulated arm 102 is provided, along a trajectory 104 that is oriented along a direction that is substantially perpendicular to the craniocaudal axis and to the sagittal plane of the patient 101 and at this point one proceeds with the opening of a percutaneous anterolateral access by means of a scalpel.

As an alternative, said articulated arm 102 can be fixed to the spinosa of the patient 101 or to the bars of the arthrodesis, if they are present.

In greater detail, the guiding element 103, which is supported by the articulated arm 102 at the movable end of the of the latter, thus can be positioned by means of the articulated arm 102 with respect to the intervertebral disc 1 to be operated at least along three degrees of freedom that are substantially parallel, respectively to the craniocaudal axis, to the sagittal axis and to the latero-lateral axis of the patient 101.

One then proceeds with the insertion in the patient 101, through the percutaneous anterolateral access 2, of a series of surgical instruments adapted to prepare the intervertebral disc 1 to accommodate an intervertebral prosthesis 3a, as well as the intervertebral prosthesis 3a.

According to the invention, these steps of insertion, which provide for the aid of the guiding element 103 in order to support and guide the surgical instruments required for the surgical procedure during its execution, are performed with the aid of a guiding wire 105, also supported by the guiding element 103, inserted previously in the patient 101 through the percutaneous anterolateral access 2 along a direction that is substantially perpendicular to the craniocaudal axis and to the sagittal plane of the patient 101, i.e., along the operating trajectory 104 described earlier.

Accordingly, as will be described in greater detail hereinafter, the cited surgical instruments and the intervertebral prosthesis 3a itself are slidingly associable with the guiding wire 105 in order to perform the surgical procedure in a wire-guided manner.

In greater detail, as in FIGS. 5 and 6, the step of insertion of the surgical instruments comprises the insertion of at least one cannulated instrument 106, which is provided internally with a removable plug element 107, until the intervertebral disc 1 is reached.

In order to render such step of insertion as minimally invasive as possible, it can be carried out by inserting such cannulated instrument 106, provided internally with the removable plug element 107, until the psoas muscle is reached; after which it is extracted, again through the percutaneous anterolateral access 2, by extracting it from the plug element 107 which remains stationary, and a series of further cannulated instruments, not shown for the sake of graphic simplicity, of progressively increasing diameter are inserted and extracted in sequence along the plug element 107, making each one of these advance together with the plug element 107 until the psoas muscle is reached, in each instance moving the soft structures interfering with the operating trajectory 104 and thus creating an operating channel.

Once such operating channel is created, one proceeds with the reinsertion of the cannulated instrument 106 until the intervertebral disc 1 is reached.

One then moves on to the removal of the plug element 107 in order to be able to insert, as shown in FIGS. 6 and 8, the guiding wire 105 within the cannulated instrument 106 and penetrate the intervertebral disc 1 for a depth that is shallower than the transverse thickness of the intervertebral disc 1.

Once the guiding wire 105 has been inserted, the cannulated instrument 106 is extracted through the percutaneous anterolateral access 2, sliding it off the guiding wire 105, which remains stationary along the operating trajectory 104, as shown in FIG. 9.

Advantageously, the cannulated instrument 106 and the plug element 107 are beveled at their distal tips 108 so as to not damage any nervous structures that might be present.

Then, as shown in FIGS. 10 to 12, one proceeds with the wire-guided insertion of at least one hernia clamp 109 provided with an external cannulation 110 that can be associated slidingly with the guiding wire 105 eccentrically with respect to the longitudinal axis of said hernia clamp 109.

Advantageously, by virtue of the ability to rotate and translate around and along the guiding wire 105, with the hernia clamp 109 it is possible to remove part of the intervertebral disc 1 in order to create a receptacle 4 for the intervertebral prosthesis 3 that is delimited by the vertebral endplates 5 and 6 that are adjacent to the intervertebral disc 1.

Once the operation has taken place, one proceeds with the extraction of the hernia clamp 109 through the percutaneous anterolateral access 2, extracting it from the guiding wire 105, which remains stationary along the operating trajectory 104.

Then, as shown in FIGS. 13 to 15, one proceeds with the wire-guided insertion of at least one cannulated rasp 111, for example of the motorized type, in order to be able to remove the cartilage of the vertebral endplates 5 and 6 with bleeding thereof so as to facilitate bone fusion between the intervertebral prosthesis 3 and the vertebral endplates 5 and 6.

Once the operation has taken place, one proceeds with the extraction of the cannulated rasp 111 through the percutaneous anterolateral access 2, sliding it off the guiding wire 105, which remains stationary along the operating trajectory 104.

Then, as shown in FIGS. 16 to 18, one proceeds with the wire-guided insertion, where the intervertebral prosthesis 3a will be positioned, and with the wire-guided extraction of said series of cannulated measurers 112 having different dimensions, which have, at their distal part, substantially the shape of a parallelepiped with radiused edges and a substantially rectangular transverse cross-section, in sequence with respect to each other so as to be able to determine the height of the intervertebral prosthesis 3a.

These cannulated measurers 112 are inserted, within the intervertebral disc 1, preferably so that their lower transverse thickness is substantially oriented along the craniocaudal axis of the patient 101, so that if a compression of the intervertebral disc 1 has occurred, following a 90° rotation of one of the cannulated measurers 112 as shown in FIGS. 19 and 20, it can be positioned so that its greater transverse thickness is substantially oriented along the craniocaudal axis of the patient 101 for the mutual spacing of the vertebral endplates 5 and 6, with consequent restoring of the intervertebral space between said intervertebral endplates 5 and 6.

Once the operation has taken place, one proceeds with the extraction of the cannulated measurer 112 through the percutaneous anterolateral access 2, extracting it from the guiding wire 105, which remains stationary along the operating trajectory 104.

Then, as shown in FIGS. 21 to 28, one proceeds with the wire-guided insertion of at least one insertion instrument 113, which carries, at its distal part, the intervertebral prosthesis 3a in such a manner as to position it correctly in the previously prepared intervertebral space.

In greater detail, the intervertebral prosthesis 3a, which as will described in greater detail hereinafter is of the wire-guided type, is associated detachably with the insertion instrument 113 by shape mating so that it can be disengaged from the insertion instrument 113, once it has been placed within the intervertebral space, for the extraction of the insertion instrument 113 and of the guiding wire 105.

As shown in FIGS. 29 and 30, if there are bone structures 8 that interfere with the operating trajectory 104 imposed by the guiding element 103 in the direction of the intervertebral disc 1, such as for example the iliac crest, prior to the step of insertion of the cannulated instrument 106 it is possible to provide for the insertion of at least one milling tool 114, in a guided manner by means of the guiding element 103, through the percutaneous anterolateral access 2, in order to mill the bone structure 8 in order to create a passage through which the cannulated instrument 106 is then inserted.

Conveniently, said milling tool 114 is inserted, with its bit protected by an extractable sheath, so that the bit, provided with a cutting edge, does not create lacerations during passage through the muscles but simply parts the fibers until it reaches the bone structure 8 to be operated.

Once the operation has taken place, one proceeds with the extraction of the milling tool 114 through the percutaneous anterolateral access 2.

Conveniently, the steps of insertion and/or extraction of the milling tool 114, of the surgical instruments 106, 109, 111, 112 and 113, of the guiding wire 105 and of the intervertebral prosthesis 3 can be monitored at least partially by means of second radiographs taken with the aid of the radiological device cited earlier.

Moreover, there can be a neurological device, not shown for the sake of graphical simplicity, with one pole that can be connected electrically to the surgical instruments and with other pole that can be connected to the nervous system of the patient 101 so as to warn the surgeon if the surgical instrument being used is proximate to the nervous structures of the patient 101.

In summary, the surgical method described above can utilize therefore an apparatus that comprises:
an operating table 100, on which the patient 101 to be operated is rested;
surgical instruments required for the surgical procedure;
a radiological device adapted to take snapshots of radiographs in order to determine the exact position of the intervertebral disc 1 on which to operate and the operating trajectory 104 for guiding the surgical instruments by checking for the presence of intestinal loops or loops of the peritoneum along said operating trajectory 104 so as to avoid the tearing or passing through thereof;
a guiding wire 105, which can be inserted in the patient 101 through a percutaneous anterolateral access 2 along a direction that is substantially perpendicular to the craniocaudal axis and to the sagittal plane of the patient 101, in such a manner that the surgical instruments can be associated slidingly with the guiding wire 105 to perform the surgical procedure in a wire-guided manner.

Advantageously, the guiding wire 105 can of the type divided into centimeters, i.e., it can have a preset length so as to be able to limit the use of the radiological device only to the initial steps of the operation, since if is known where the guiding wire 105 is positioned in the intervertebral disc 1 by means of the first radiograph and it is known how far the surgical instrument being used has translated with respect to said guiding wire 105, even without having visual confirmation there is always certainty as to where the surgical instrument being used is operating.

Furthermore, in order to prevent the advancement of the guiding wire 105 during the sliding of the surgical instruments thereon, this can be provided with a plurality of pawls adapted to engage with the organic tissue of the patient 101 to be operated.

Conveniently, such pawls, which can be made of an elastic material such as for example an alloy of nickel and titanium, are of the type that can be closed, thus acting as a unidirectional retention element for extracting the guiding wire 105 from the patient 101 to be operated.

As regards the previously mentioned surgical instruments, they comprise:
at least one scalpel at the opening of the percutaneous anterolateral access 2;
at least one cannulated instrument 106, which is adapted to be inserted in the patient 101 through the percutaneous anterolateral access 2 along a direction that is substantially perpendicular to the craniocaudal axis and to the sagittal plane of the patient 101.

Advantageously, as already noted, the cannulated instrument 106 is beveled at its distal tip so as to avoid damaging any nervous structures that might be present and is provided internally with a plug element 107 that is beveled at its distal tip so as to avoid damaging any nervous structures that might be present and is removable for the insertion of the guiding wire 105 once the cannulated instrument 106 has been positioned proximate to the intervertebral disc 1.

Furthermore, said surgical instruments comprise:
at least one hernia clamp 109, provided with an external cannulation 110 that is slidingly associable with the guiding wire 105 in an eccentric manner with respect to the longitudinal axis of said hernia clamp 109 for the wire-guided of the latter in the patient 101 and to remove the part of the intervertebral disc 1 in the place of which the intervertebral prosthesis 3a will be placed, by rotating around the guiding wire 105;
at least one cannulated rasp 111, for example of the motorized type, which can be associated slidingly with the guiding wire 105 for its wire-guided insertion in the patient 101 so as to be able to remove the cartilage of the vertebral endplates 5 and 6 that are adjacent to the intervertebral disc 1 and cause their bleeding so as to facilitate bone fusion between the intervertebral prosthesis 3a and said vertebral endplates 5 and 6;
at least one cannulated measurer 112, which can be associated slidingly with the guiding wire 105 for its wire-guided insertion in the patient 101 so as to be able to determine the height of the intervertebral prosthesis 3a to be implanted.

Advantageously, as already noted, the cannulated measurer 112 has, at its distal part, substantially the shape of a parallelepiped with radiused edges and a substantially rectangular transverse cross-section, so as to be inserted within the intervertebral disc 1, where the intervertebral prosthesis 3a will be positioned, so that its lower transverse thickness is substantially oriented along the craniocaudal axis of the patient 101.

In greater detail, the cannulated measurer 112 can rotate about the guiding wire 105 in such a manner as to be able to restore the intervertebral space between the intervertebral endplates 5 and 6, being able to position it so that its greater transverse thickness is substantially oriented along the craniocaudal axis of the patient 101 as a consequence of a 90° rotation of said cannulated measurer 112.

Furthermore, said surgical instruments comprise at least one insertion instrument 113, which can be associated at its distal part with the intervertebral prosthesis 3a to be implanted, is essentially shaped like a cannula and is slidingly associable with the guiding wire 105 for its wire-guided insertion in the patient 101 so as to be able to position correctly the intervertebral prosthesis 3a.

Advantageously, said insertion instrument 113 has, at its distal part, a threaded shank 115 that can engage in a threaded hole 7, which is formed in the intervertebral prosthesis 3a at a side wall of the intervertebral prosthesis 3a substantially coaxially to a through hole 9 that passes through the intervertebral prosthesis 3a from side to side along a direction that is perpendicular to the sagittal plane of the patient 101 and is adapted to accommodate slidingly the guiding wire 105, so as to be able to move transversely the intervertebral prosthesis 3a within the intervertebral space and so that it can be unscrewed from the intervertebral prosthesis 3a, so that it can be removed from the patient 101.

Conveniently, the threaded hole 7 has a larger diameter than the through hole 9.

To complete the surgical instruments, they comprise:
at least one milling tool 114, which can be inserted in the patient 101 through the percutaneous anterolateral access 2 and is adapted to create a through hole through any bone structures, such as for example the iliac crest 8, that interfere with the operating trajectory 104;
an articulated arm 102, for example of the motorized type, which can be fixed to the operating table 101 or to the spinosa of the patient 101 or to the bars of the arthrodesis, if they are present, and is provided, at its movable end, with a guiding element 103 that is adapted to support and guide the surgical instruments described so far and to support the guiding wire 105 during the surgical procedure;
a neurological device with one pole that can be connected electrically to the surgical instruments and with the other pole that can be connected to the nervous system of the patient 101 in such a manner as to warn the surgeon if the surgical instrument being used is proximate to the nervous structures of the patient 101.

In greater detail, as already mentioned, the guiding element 103 can be positioned by means of the articulated arm 102, with respect to the intervertebral disc 1 to be operated, at least along three degrees of freedom that are substantially parallel respectively to the craniocaudal axis, to the sagittal axis and to the latero-lateral axis of the patient 101.

With particular reference to FIGS. 21 to 28, as regards the intervertebral prosthesis 3a, which can be made of osteoconductive material so as to facilitate fusion with the adjacent vertebrae 11 and 12 between which it is inserted, as shown in FIG. 24, in a first embodiment thereof said prosthesis comprises an element 10a that has a substantially disc-like shape and is adapted to be inserted between two adjacent vertebrae 11 and 12 of the patient 101 instead of the intervertebral disc 1 comprised between them, so as to support entirely the vertebral endplates over the largest possible surface and simultaneously provide an anteroposterior angle that is aimed at maintaining physiological lordosis and sagittal balance in order to form a bone bridge between the adjacent vertebrae 11 and 12.

As already introduced previously, the element 10a has a through hole 9 that passes through it from side to side and is adapted to accommodate slidingly the guiding wire 105, previously inserted in the patient 101 along a direction that is perpendicular to the sagittal plane of the patient 101 through a percutaneous anterolateral access 2, for the wire-guided insertion of the intervertebral prosthesis 3a.

Conveniently, the through hole 9 is extended along a radial direction with respect to the geometry of the element 10a so that it is oriented, once implanted, along a direction that is substantially perpendicular to the craniocaudal axis and to the sagittal plane of the patient 101.

In this manner, the threaded hole 7, which is formed at a side wall of the element 10a, is substantially coaxial to the through hole 9, with a larger diameter than the through hole 9.

Furthermore, the element 10a has, at its upper face 14 and at its lower face 15 which are intended to make contact with the vertebral endplates 5 and 6 of the adjacent vertebrae 11 and 12, a surface that is provided with a plurality of protruding bodies 17, which consist for example of toothed ridges and are adapted to facilitate the grip of the intervertebral prosthesis 3a with the vertebral endplates 5 and 6.

Finally, the element 10a has at least one lightening cavity 18 that passes through it from the upper face 14 to the lower face 15.

With particular reference to FIGS. 31 to 40, in a second embodiment of the intervertebral prosthesis, generally designated with the reference numeral 3b, which in part has the same characteristics as the intervertebral prosthesis 3a, it comprises means of activating at least one between means of radial expansion of the element that defines the structure of the intervertebral prosthesis 3b, at least according to a direction that is parallel to the axis of the through hole 9 in order to support the vertebral endplates 5 and 6 along the entire transverse space occupation thereof, and means of stabilizing the intervertebral prosthesis 3b to the vertebral endplates 5 and 6 of the adjacent vertebrae 11 and 12 for the self-stabilization of the intervertebral prosthesis 3b.

In more detail, the element that defines the structure of the intervertebral prosthesis 3b, generally designated with the reference numeral 10b, comprises at least two separate portions 20 and 21 that can move with respect to each other along a direction that is substantially parallel to the axis of the through hole 9 by means of a shape mating.

Furthermore, advantageously, the above mentioned means of radial expansion comprise at least one leadscrew coupling associated with the two separate portions 20 and 21 in such a manner as to allow their mutual spacing apart following the rotation of the screw with respect to the female thread.

More specifically, said leadscrew coupling comprises a screw 23 that is rotatably supported by one of the two separate portions 21 coaxially with the through hole 9 and at least one female thread associated integrally with the other separate portion 20 coaxially with the through hole 9.

Conveniently, the separate portion 21 which rotatably supports the above-mentioned screw is intended to be inserted last through the percutaneous anterolateral access 2.

Advantageously, both the screw 23 and the female thread are of the hollow type in order to allow wire-guided insertion of the intervertebral prosthesis 3b.

Considering the above-mentioned activation means, with particular reference to FIGS. 34a and 34b, these comprise engagement means which are defined at least on the free end of the screw and are associable with a screwer instrument for rotating the screw with respect to the separate portion 21 and to the female thread.

Again with particular reference to FIGS. 34a and 34b, the stabilizing means on the other hand comprise a plurality of pointed appendages 24 which can move between an inactive position, in which the pointed appendages 24 are completely accommodated within the element 10b as shown in FIG. 34a, and an engaged position, in which the pointed appendages 24 protrude from the element 10b in the direction of the vertebral endplates 5 and 6.

In more detail, such pointed appendages 24 are rotatably supported by the two separate portions 20 and 21 about pivoting axes 25 that are substantially perpendicular to the rotation axis of the screw 23 and are oriented so as to be substantially parallel to the intervertebral endplates 5 and 6.

Furthermore, each pointed appendage 24 has, on the end portion pivoted to the two separate portions 20 and 21, a toothed profile 26 adapted to engage with the crests of the screw 23 in order to rotate the pointed appendages 24 about the pivoting axes 25 following the rotation of the screw 23.

With particular reference to FIGS. 37 and 38, the association between such screwer instrument and the screw 23 can be achieved for example by way of a guiding wire 105 of the profiled type which is adapted to engage by shape mating with the hollow part of the screw and with the screwer instrument itself.

In this way, such screwer instrument can comprise a grip 116 to be associated with the guiding wire 105 externally to the patient 101 so as to allow the rotation of the guiding wire 105 and as a consequence the rotation of the screw for the radial expansion of the element 10b and the simultaneous egress of the pointed appendages 24.

Alternatively, such screwer instrument can comprise a substantially cannula-shaped body, which can be slidingly associated with the guiding wire 105 for its wire-guided insertion inside the patient 101 and can be engaged at its distal end with engagement means defined by the screw 23 so that it can activate at least one between means of radial expansion and means of stabilization of said intervertebral prosthesis.

Conveniently, in this case the screwer instrument is dissociable from the intervertebral prosthesis 3b so that it can be removed once at least one between the means of radial expansion and the stabilizing means have been activated.

As a consequence, if an intervertebral prosthesis 3b of this type is used, then the surgical method will also have a further step added, after the step of insertion of the intervertebral prosthesis 3b and before the step of removal of the guiding wire 105.

More specifically, such step would consist of the activation of the intervertebral prosthesis 3b by way of association of the screwer instrument with the activation means of at least one between means of radial expansion of the intervertebral prosthesis 3b, at least along a direction parallel to the axis of the through hole 9 for supporting the vertebral endplates 5 and 6 along the entire transverse space occupation thereof, and means of stabilizing the intervertebral prosthesis 3b to the vertebral endplates 5 and 6 of the adjacent vertebrae 11 and 12 for the self-stabilization of the intervertebral prosthesis 3b.

Subsequently, the screwer instrument is dissociated from the intervertebral prosthesis 3b, with the prosthesis arranged within the intervertebral space, for the extraction of the insertion instrument and of the guiding wire 105.

In practice it has been found that the intervertebral prosthesis, the apparatus for implanting intervertebral prostheses and the surgical method for implanting intervertebral prosthesis, particularly for percutaneous procedures for mini-invasive surgery, according to the invention, achieve fully the intended aim and objects, since they allow to achieve an intervertebral fusion adapted to create a bone bridge between two adjacent vertebrae with a percutaneous mini-invasive surgical procedure.

In particular, by virtue of the fact that no retraction or distraction instruments are required but the intermediate structures (muscles) are crossed with the instruments and ultimately with the implant, it is possible to avoid problems related to stretching or compression of the femoral plexus.

Passage through the muscles in fact lasts only a few seconds for each instrument and the maximum size of the passage is the size of the prosthesis, which in the maximum size is 15 by 22 mm. Furthermore, all the instruments and the implant itself are slender in order to avoid tearing the structures through which they pass.

Another advantage of the intervertebral prosthesis, of the apparatus and of the surgical method according to the present invention resides in that by virtue of the fact that the patient is placed in a prone position the aorta and vena cava vascular structures and the abdominal organs in a prone position presumably by gravity move away from the operating corridor of the lateral access pathway.

A further advantage of the intervertebral prosthesis, of the apparatus and of the surgical method according to the present invention resides in that the anulus is not removed but only a small initial opening is created; since the structure of the anulus is furthermore made of X-crossed fibers, the subsequent passages tend to open them without tearing them; this allows, once the implant has been inserted, the fibers of the anulus that have been crossed to close on themselves, in turn containing the implant and avoiding its dislocation.

Another advantage of the intervertebral prosthesis, of the apparatus and of the surgical method according to the present invention resides in that with the prone position of the patient (with respect to the position on one side of the background art) it is possible, by means of maneuvers with the table of the patient, to restore the correct sagittal balance intraoperatively.

Another advantage of the intervertebral prosthesis, of the apparatus and of the surgical method according to the present invention resides in that, the intervertebral prosthesis being adjustable in size once implanted, it is possible to operate with a minimum of space occupation of the prostheses in order to then re-adapt it with extreme precision to the vertebral dimensions of the disk in which it is implanted.

Another advantage of the intervertebral prosthesis, of the apparatus and of the surgical method, according to the present invention, consists in that the intervertebral prosthesis is provided with self-stabilizing elements that prevent unwanted migrations of the prostheses.

Another advantage of the intervertebral prosthesis, of the apparatus and of the surgical method according to the present invention resides in that it is possible to position the implant at the L5-S1 level, currently being the only implant of this type that can be implanted at this level.

A further advantage of the intervertebral prosthesis, of the apparatus and of the surgical method according to the present invention resides in that the execution times of the procedure are distinctly shorter than the background art, and in the case of posterior arthrodesis are reduced by approximately 50%.

The intervertebral prosthesis, the apparatus for implanting intervertebral prostheses and the surgical method for implanting intervertebral prostheses, particularly for percutaneous mini-invasive surgery procedures, thus conceived are susceptible of numerous modifications and variations, all of which are within the scope of the accompanying claims.

Furthermore, all the details may be replaced with other technically equivalent elements.

In practice, the materials used, as well as the contingent shapes and dimensions, may be any according to the requirements and the state of the art.

The disclosures in U.S. patent application Ser. No. 14/657,803 from which this application claims priority are incorporated herein by reference.

Where technical features mentioned in any claim are followed by reference signs, those reference signs have been included for the sole purpose of increasing the intelligibility of the claims and accordingly, such reference signs do not have any limiting effect on the interpretation of each element identified by way of example by such reference signs.

1.-41. (canceled)

42. An intervertebral prosthesis, particularly for percutaneous mini-invasive surgery procedures, comprising:
an element that has a substantially disc-like shape and is adapted to be inserted between two adjacent vertebrae of a patient in which the intervertebral prosthesis is to be implanted as a replacement of the intervertebral disc comprised between them, so as to support entirely the vertebral endplates over the largest possible surface and at the same time impart an anteroposterior angle aimed at maintaining physiological lordosis and sagittal balance in order to form a bone bridge between the adjacent vertebrae, wherein the element has a through hole that passes through the element from side to side and is adapted to accommodate slidingly a guiding wire, inserted beforehand in the patient along a direction that is perpendicular to a sagittal plane of the patient through a percutaneous anterolateral access, for the wire-guided insertion of the intervertebral prosthesis, the through hole being extended along a radial direction with respect to the geometry of the element so that it is oriented, once implanted, along a direction that is substantially perpendicular to a craniocaudal axis of the patient and to the sagittal plane.

43. The intervertebral prosthesis according to claim 42, wherein the element has a threaded hole that is formed at a side wall of the element substantially coaxially to the through hole, the threaded hole having a larger diameter than the through hole for its engagement with a threaded shank that is formed at the end of an insertion instrument that is substantially shaped like a cannula, so that it can be wire-guided during its insertion in the patient by means of the guiding wire.

44. The intervertebral prosthesis according to claim 43, wherein the element has, at an upper face thereof and at a lower face thereof intended to make contact with the vertebral endplates of the adjacent vertebrae, a surface that is provided with a plurality of protruding bodies adapted to facilitate the grip of the intervertebral prosthesis with the vertebral endplates of the adjacent vertebrae.

45. The intervertebral prosthesis according to claim 44, wherein the protruding bodies comprise toothed ridges.

46. The intervertebral prosthesis according to claim 43, comprising means of activating at least one between means of radial expansion of the element, at least according to a direction that is parallel to the axis of the through hole in order to support the vertebral endplates along the entire transverse space occupation thereof, and stabilizing means for stabilizing the intervertebral prosthesis to the vertebral endplates of the adjacent vertebrae for the self-stabilization of the intervertebral prosthesis.

47. The intervertebral prosthesis according to claim 46, wherein the element comprises at least two separate portions that can move with respect to each other along a direction that is substantially parallel to the axis of the through hole by means of a shape mating and wherein the means of radial expansion comprise at least one leadscrew coupling associated with the at least two portions in such a manner as to allow their mutual spacing apart following the rotation of the screw with respect to the female thread of the at least one leadscrew coupling.

48. The intervertebral prosthesis according to claim 47, wherein the leadscrew coupling comprises a screw that is rotatably supported by one of the at least two portions coaxially with the through hole and at least one female thread associated integrally with the other one of the at least two portions coaxially with the through hole, the separate portion which rotatably supports the screw being intended to be inserted last through the percutaneous anterolateral access and the screw and the at least one female thread being of the hollow type in order to allow the wire-guided insertion of the intervertebral prosthesis.

49. The intervertebral prosthesis according to claim 48, wherein the activation means comprise engagement means which are defined at least on the free end of the screw and are associable with a screwer instrument for rotating the screw with respect to the separate portion and to the at least one female thread.

50. The intervertebral prosthesis according to claim 46, wherein the stabilizing means comprise a plurality of pointed appendages that can move between an inactive position, in which the pointed appendages are completely accommodated within the element, and an engaged position, in which the pointed appendages protrude from the element in the direction of the vertebral endplates, the pointed appendages being rotatably supported by the at least two portions about pivoting axes that are substantially perpendicular to the rotation axis of the screw and are oriented so as to be substantially parallel to the intervertebral endplates, each one of the pointed appendages having, on the end portion pivoted to the at least two separate portions, a toothed profile adapted to engage with the crests of the screw in order to rotate the pointed appendages about the pivoting axes following the rotation of the screw.

51. The intervertebral prosthesis according to claim 44, wherein the element has at least one lightening cavity that passes through the element from the upper face to the lower face.

52. The intervertebral prosthesis according to claim 47, wherein the element has a trabecular structure made of osteoconductive material so as to facilitate bone fusion between the intervertebral prosthesis and the adjacent vertebrae.

53. An apparatus for implanting intervertebral prostheses, particularly for percutaneous mini-invasive surgery procedures, comprising:
an operating table on which the patient to be operated is rested;
surgical instruments required for the surgical procedure;
a radiological device adapted to take snapshots of radiographs in order to determine the exact position of the intervertebral disc on which to operate and the operating trajectory for guiding the surgical instruments by checking for the presence of intestinal loops or loops of the peritoneum along the operating trajectory so as to avoid the tearing or passing through thereof;
further comprising at least one guiding wire that can be inserted in the patient through a percutaneous anterolateral access along a direction that is substantially perpendicular to the craniocaudal axis and to the sagittal plane of the patient, the surgical instruments being slidingly associable with the guiding wire to perform the surgical procedure in a wire-guided manner.

54. The apparatus according to claim 53, wherein the guiding wire is of the type divided into centimeters.

55. The apparatus according to claim 52, wherein the guiding wire comprises a plurality of pawls adapted to engage with the organic tissue of the patient to be operated and prevent the advancement of the guiding wire during the sliding of the surgical instruments on the guiding wire, the pawls being of the type that can be closed for the extraction of the guiding wire from the patient to be operated.

56. The apparatus according to claim 53, wherein the surgical instruments comprise at least one scalpel at the opening of the percutaneous anterolateral access.

57. The apparatus according to claim 53, wherein the surgical instruments comprise at least one cannulated instrument (106) that is adapted to be inserted in the patient through the percutaneous anterolateral access along a direction that is substantially perpendicular to the craniocaudal axis and to the sagittal plane, the cannulated instrument being beveled at its distal tip so as to not damage any nervous structures that might be present and being provided internally with a plug element that is beveled at its distal tip so as to not damage any nervous structures that might be present and is removable for the insertion of the guiding wire once the cannulated instrument has been positioned proximate to the intervertebral disc.

58. The apparatus according to claim 53, wherein the surgical instruments comprise at least one hernia clamp which is provided with an external cannulation that can be associated slidingly with the guiding wire in an eccentric manner with respect to the longitudinal axis of the at least one hernia clamp for the wire-guided insertion of the at least one hernia clamp within the patient and to remove a part of the intervertebral disc, in the place of which an intervertebral prosthesis will be placed, by rotating about the guiding wire.

59. The apparatus according to claim 53, wherein the surgical instruments comprise at least one cannulated rasp that can be associated slidingly with the guiding wire for its wire-guided insertion within the patient so as to be able to remove the cartilage of the vertebral endplates that are adjacent to the intervertebral disc and cause their bleeding, so as to facilitate bone fusion between the intervertebral prosthesis and the vertebral endplates.

60. The apparatus according to claim 59, wherein the at least one cannulated rasp is of the motorized type.

61. The apparatus according to claim 53, wherein the surgical instruments comprise at least one cannulated measurer that can be associated slidingly with the guiding wire for its wire-guided insertion within the patient so as to be able to determine the height of the intervertebral prosthesis to be implanted.

62. The apparatus according to claim 61, wherein the at least one cannulated measurer has, at its distal part, substantially the shape of a parallelepiped with rounded edges and a substantially rectangular transverse cross-section, so as to be inserted within the intervertebral disc, where the intervertebral prosthesis will be positioned, so that its lower transverse thickness is substantially oriented along the craniocaudal axis, the cannulated measurer being able to rotate around the guiding wire so as to be able to restore the intervertebral space between the intervertebral endplates, positioning the at least one cannulated measurer with its greater transverse thickness oriented substantially along the craniocaudal axis following a 90° rotation of the cannulated measurer.

63. The apparatus according to claim 53, wherein the surgical instruments comprise at least one insertion instrument that can be associated at its distal part with the intervertebral prosthesis to be implanted, is shaped substantially like a cannula and can be associated slidingly with the guiding wire for its wire-guided insertion within the patient so as to be able to position the intervertebral prosthesis correctly, the at least one insertion instrument being disengageable from the intervertebral prosthesis so that it can be removed once the intervertebral prosthesis has been placed within the intervertebral space.

64. The apparatus according to claim 63, wherein the at least one insertion instrument has, at the distal part thereof, a threaded shank that can engage in a threaded hole, which is formed in the intervertebral prosthesis at a side wall of the intervertebral prosthesis substantially coaxially to a through hole that passes through the intervertebral prosthesis from side to side along a direction that is perpendicular to the sagittal plane and is adapted to accommodate slidingly the guiding wire, so as to be able to move transversely the intervertebral prosthesis within the intervertebral space and so that it can be unscrewed from the intervertebral prosthesis so that it can be removed from the patient, the threaded hole having a larger diameter than the through hole.

65. The apparatus according to claim 53, wherein the surgical instruments comprise at least one screwer instrument that can be associated with engagement means defined by a screw that is associated with the intervertebral prosthesis to be implanted so that it can activate at least one between means of radial expansion and stabilizing means of the intervertebral prosthesis, the at least one screwer instrument being dissociable from the intervertebral prosthesis so that it can be removed once at least one between the means of radial expansion and the stabilizing means have been activated.

66. The apparatus according to claim 53, wherein the surgical instruments comprise at least one milling tool that can be inserted in the patient through the percutaneous anterolateral access and is adapted to create a through hole through any bone structures that interfere with the operating trajectory.

67. The apparatus according to claim 53, further comprising an articulated arm which can be fixed to the operating table or to the spinosa of the patient or to the bars of the arthrodesis, if they are provided, the articulated arm being provided, at its movable end, with a guiding element that is adapted to support and guide the surgical instruments and to support the guiding wire during the surgical procedure, the guiding element being arrangeable, by means of the articulated arm with respect to the intervertebral disc to be operated, at least along three degrees of freedom that are substantially parallel, respectively, to the craniocaudal axis, to the sagittal plane and to the latero-lateral axis of the patient.

68. The apparatus according to claim 67, wherein the articulated arm is of the motorized type.

69. The apparatus according to claim 53, further comprising a neurological device with one pole that can be connected electrically to the surgical instruments and with the other pole that can be connected to the nervous system of the patient so as to warn the surgeon if the surgical instrument being used is proximate to the nervous structures of the patient.

70. A surgical method for implanting intervertebral prostheses, particularly for percutaneous mini-invasive surgery procedures, comprising:
placing a patient to be operated on an operating table;
taking a first radiograph by means of a radiological device in order to establish the exact position of the intervertebral disc to be operated;
opening a percutaneous anterolateral access by means of a scalpel;
inserting in the patient, through the percutaneous anterolateral access, a series of surgical instruments adapted to prepare the intervertebral disc to accommodate an intervertebral prosthesis;
inserting the intervertebral prosthesis in the patient through the percutaneous anterolateral access;
wherein the insertion steps are performed with the aid of a guiding wire, inserted previously in the patient through the percutaneous anterolateral access along a direction that is substantially perpendicular to the craniocaudal axis and to the sagittal plane of the patient, the surgical instruments and the intervertebral prosthesis being associable slidingly with the guiding wire to perform the surgical procedure in a wire-guided manner.

71. The surgical method according to claim 70, comprising a further step of pre-operative radiology adapted to define the operating trajectory for guiding the surgical instruments by checking for the presence of intestinal loops or loops of the peritoneum along the operating trajectory so as to avoid the tearing or passing through thereof.

72. The surgical method according to claim 70, wherein, prior to the insertion steps, an articulated arm is fixed to the operating table or to the spinosa of the patient or to the bars of the arthrodesis, if they are present, in such a manner as to position a guiding element, with which the articulated arm is provided, which is adapted to support and guide the surgical instruments and to support the guiding wire during the surgical procedure, the guiding element defining an operating trajectory that is oriented along a direction that is substantially perpendicular to the craniocaudal axis and to the sagittal plane.

73. The surgical method according to claim 70, wherein, prior to the step of insertion of the series of surgical instruments and if there are bone structures that interfere with the operating trajectory imposed by the guiding element in the direction of the intervertebral disc, the following are provided:
the insertion of at least one milling tool, in a guided manner by means of the guiding element through the percutaneous anterolateral access;
the milling of the bone structure to create a passage through which the cannulated instrument is to be inserted;
the extraction of the at least one milling tool through the percutaneous anterolateral access.

74. The surgical method according to claim 70, wherein the step of insertion of the series of surgical instruments comprises:
the insertion of at least one cannulated instrument, provided internally with a removable plug element, until the intervertebral disc is reached, the at least one cannulated instrument and the plug element being beveled at their distal tips so as to not damage any nervous structures that might be present, the at least one cannulated instrument being inserted with the aid of the guiding element along the operating trajectory;
the removal of the plug element;
the insertion of the guiding wire within the cannulated instrument and the penetration of the intervertebral disc on the part of the guiding wire for a depth that is less than the transverse thickness of the intervertebral disc;
the extraction of the cannulated instrument through the percutaneous anterolateral access, extracting it from the guiding wire, which remains stationary.

75. The surgical method according to claim 74, wherein the step of insertion of the at least one cannulated instrument, provided internally with the plug element, comprises:
the insertion of the cannulated instrument, provided internally with the plug element, until the psoas muscle is reached,
the extraction of the cannulated instrument through the percutaneous anterolateral access by extracting it from the plug element which remains stationary,
the insertion and extraction in sequence of a series of further cannulated instruments of progressively increasing diameter along the plug element, making each one of the cannulated instruments advance together with the plug element until the psoas muscle is reached, in each instance moving the soft structures interfering with the operating trajectory and thus creating an operating channel,
the reinsertion of the cannulated instrument until the intervertebral disc is reached.

76. The surgical method according to claim 70, wherein the step of insertion of the series of surgical instruments comprises:
the wire-guided insertion of at least one hernia clamp provided with an external cannulation that can be associated slidingly with the guiding wire in an eccentric manner with respect to the longitudinal axis of the at least one hernia clamp,
the removal of part of the intervertebral disc with the aid of the at least one hernia clamp with rotation of the at least one hernia clamp around the guiding wire to create a receptacle for the intervertebral prosthesis that is delimited by the vertebral endplates that are adjacent to the intervertebral disc,
the extraction of the at least one hernia clamp through the percutaneous anterolateral access, extracting it from the guiding wire, which remains stationary.

77. The surgical method according to claim 70, wherein the step of insertion of the series of surgical instruments comprises:
the wire-guided insertion of at least one cannulated rasp,
the removal, with the aid of the at least one cannulated rasp, of the cartilage of the vertebral endplates with bleeding thereof in such a manner as to facilitate bone fusion between the intervertebral prosthesis and the vertebral endplates,
the extraction of the at least one cannulated rasp through the percutaneous anterolateral access, extracting it from the guiding wire, which remains stationary.

78. The surgical method according to claim 70, wherein the step of insertion of the series of surgical instruments comprises:
the wire-guided insertion, where the intervertebral prosthesis will be positioned, and the wire-guided extraction of a series of cannulated measurers of different sizes, having a shape, at their distal part, that is substantially like a parallelepiped with radiused edges and a substantially rectangular transverse cross-section, in succession with respect to each other so as to be able to determine the height of the intervertebral prosthesis to be implanted, the cannulated measurers being inserted within the intervertebral disc, so that their lower transverse thickness is oriented substantially along the craniocaudal axis,
if a compression of the intervertebral disc has occurred, the rotation through 90° of one of the cannulated measurers in such manner as to position it with its greater transverse thickness oriented substantially along the craniocaudal axis, for the mutual spacing of the vertebral endplates, with consequent restoring of the intervertebral space between the intervertebral endplates,
the extraction of the cannulated measurer through the percutaneous anterolateral access by extracting it from the guiding wire, which remains stationary.

79. The surgical method according to claim 70, wherein the step of insertion of the intervertebral prosthesis comprises:
the wire-guided insertion of at least one insertion instrument that supports, at its distal part, the intervertebral prosthesis so as to position correctly the intervertebral prosthesis in the previously prepared intervertebral space, the intervertebral prosthesis being wire-guided and being associated detachably with the at least one insertion instrument by shape mating,
the disengagement of the at least one insertion instrument from the intervertebral prosthesis, with the intervertebral prosthesis arranged within the intervertebral space, for the extraction of the at least one insertion instrument and of the guiding wire.

80. The surgical method according to claim 70, wherein, after the step of inserting the intervertebral prosthesis, there is a step of activation of the intervertebral prosthesis by way of:
the association of at least one screwer instrument with means of activating at least one between means of radial expansion of the intervertebral prosthesis, at least according to a direction that is parallel to the axis of the through hole in order to support the vertebral endplates along the entire transverse space occupation thereof, and means of stabilizing the intervertebral prosthesis to the vertebral endplates of the adjacent vertebrae for the self-stabilization of the intervertebral prosthesis,
the dissociation of the at least one screwer instrument from the intervertebral prosthesis, with the intervertebral prosthesis arranged within the intervertebral space, for the extraction of the at least one insertion instrument and of the guiding wire.

81. The surgical method according to claim 73, wherein the steps of insertion and/or extraction of the milling tool, of the series of surgical instruments and of the intervertebral prosthesis are at least partly monitored by means of second radiographs taken with the aid of the radiological device.

82. The surgical method according to claim 73, wherein in the steps of insertion and/or extraction of the milling tool, of the series of surgical instruments and of the intervertebral prosthesis at least the milling tool and the at least one cannulated instrument are connected electrically to a pole of a neurological device, the other pole of the neurological device being connected electrically to the nervous system of the patient so as to warn the surgeon if the surgical instrument being used is proximate to the nervous structures of the patient.
